(19) United States
(12) Patent Application Publication
(10) Pub. No.: US 2004/0058423 A1
(43) Pub. Date: Mar. 25, 2004

(54) Fast electrical lysis of cells and rapid collection of the contents thereof using capillary electrophoresis

(76) Inventors: Nancy Albritton, Irvine, CA (US); Guann-Pyng Li, Irvine, CA (US); Mark Bachman, Irvine, CA (US); Christopher Sims, Irvine, CA (US); Futian Han, Ames, IA (US)

Correspondence Address: MARK W. ROBERTS, Ph.D., DORSEY & WHITNEY LLP, Suite 3400, 1420 Fifth Avenue, Seattle, WA, 98101, US

(21) Appl. No.: 10/429,282

(22) Filed: May 2, 2003

Related U.S. Application Data
(60) Provisional application No. 60/377,476, filed on May 3, 2002.

(51) Int. Cl. C12M 1/42; C12N 13/00

(52) U.S. Cl. 435/173.7; 435/285.2; 435/288.4; 435/305.2; 435/306.1; 204/451; 204/601

(57) ABSTRACT

The invention provides apparatus and methods for subsecond lysis of selected cells in a cell chamber using a voltage pulse of 10 ms to 10 μs in duration followed by nearly simultaneous collection of the lysed cellular contents into a capillary electrophoresis tube or other suitable micro-collection device. Cell chambers and capillary electrophoresis tubes configured with electrodes for performing the electrical lysis are described. The influence of variables that govern the rate of cell lysis, such as inter-electrode distance, pulse duration, and pulse strength are also described. The methods are illustrated using fluorophores that are loaded into a cell then collected following electrical lysis, separated by electrophoresis, and then detected by laser-induced fluorescence detection in a capillary electrophoresis system.

### CROSS-REFERENCE TO RELATED APPLICATION(S)

This invention claims priority to U.S. provisional patent application No. 60/377,476 filed May 3, 2002.

### STATEMENT OF GOVERNMENT INTEREST

This invention was made with Government support under Grant No. CA91216, awarded by the National Institutes of Health. The Government has certain rights in this invention.

### TECHNICAL FIELD

The invention relates to the field of lysis and collection of material from a biological cell, particularly to cell chambers and micro-collection devices configured for electrical lysis of the cell, and more particularly for systems configured to collect the contents of the lysed cell in a capillary electrophoresis tube.

### BACKGROUND OF THE INVENTION

Capillary electrophoresis (CE) has found abundant applications for bioanalysis, especially for the identification and quantification of cellular analytes. The capillary's micron dimensions and picoliter to nanoliter sample volumes are ideal matches for examination of the contents of biological cells. A number of reviews describing the analysis of single cells with CE have been published [1-6]. In the past decade:, CE has demonstrated increasing utility for the quantitative analysis of single cells. New applications for the analysis of dynamic cellular properties using a variety of micro-collection devices including CE tubes demand methods of cellular lysis and collection with sufficient temporal resolution to accurately measure these processes. In particular intracellular signaling pathways involving many enzymes can he modulated on subsecond time scales.

One method described in U.S. Pat. Nos. 6,156,576 and 6,335,201 for combining rapid cellular lysis and collection of the contents of cells is a laser-based cell lysis system (the Laser Micropipette System [LMS]), which was developed by our lab to rapidly lyse a single cell (or cells) with rapid subsequent collection of the contents for the analysis of enzymatic activity by capillary electrophoresis The LMS, has a number of valuable advantages for performing cell-based assays. Among these are the rapid temporal resolution of the sampling method and its compatibility with optical microscopy. In the current LMS design, a capillary is mounted above a cell placed on the stage of an inverted microscope. The cell is loaded with fluorescent reporters of an enzyme activity, such as a kinase activity. At a desired time, a focused pulse from a Nd:YAG laser is used to create a shock wave which lyses the cell. The cell's contents (including the reporters) are loaded into the capillary and separated by the electrophoresis. The reporters undergo a shift in their electrophoretic mobility when acted upon by a kinase. The advantages of the LMS include i) rapid cell lysis and fast termination of cellular reactions (<<33 ms), ii) efficient sampling of cell contents, iii) applicability to both adherent and non-adherent cells, and iv) excellent reproducibility. We have shown that this method for rapid cell sampling prevents artifacts in the measurement of kinase activity compared to hypoosmotic lysis over several seconds. [12].

Unfortunately, current instrumental design and complexity limit the numbers and types of investigators that can easily utilize the laser micropipette system. One drawback is the relatively high cost of the Nd:YAG laser required for the cell lysis. Perhaps more limiting is the technical expertise required to properly align the laser through the microscope and to maintain its proper alignment over time. The current design also imposes limits on the throughput of experiments. At present, cells are cultured on a coverslip in random locations and at a low density. This is necessary because the shock wave, which rapidly lyses the cell of interest, propagates through the substrata and media lysing other nearby cells. For single-cell experiments one must search for a cell without close neighbors, which is a time-consuming process. Additionally only one measurement per cell culture chamber can be performed.

Other techniques for lysing cells include the use of capillaries to perform the lysis. In one method for studies of large, non-mammalian cells (200-1000 microns in size), a capillary tapered to a sharp point is inserted into the cell to remove a plug of cytoplasmic sample [7-9]. Other methods include detergent and/or hypoosmotic solutions to lyse mammalian cells and to release the cell's contents into a capillary for analysis. [1-6]. Mammalian cells, are smaller (typically less than about 100 microns) and in certain practices, the whole cell is injected into the capillary for analysis. In other methods, application of an electric field through the walls of a capillary has been used to lyse a cell that has been drawn into the capillary. [10].

Electrical lysis of cells has been demonstrated as a modification of various electroporation techniques. Electroporation is a phenomenon that has been studied extensively. [13-18]. Multiple electrical pulses of a defined pulse width and voltage are used to induce a potential difference across the cell. When the transmembrane potential difference is large enough, pores are formed in the membrane which rapidly seal upon removal of the electric field. This process is widely used to load exogenous molecules into cells. A number of innovative strategies have been developed to rapidly and reversibly electroporate single, adherent cells. Owar and colleagues demonstrated that an electrolyte-filled capillary or miniaturized carbon-fiber electrodes could selectively electroporate single cells or portions of adherent cells [23, 24]. Haas et al utilized a pulled glass pipette to porate single cells within the brains of living animals. [51]. In addition a number of investigators have used modified patch clamp methods to electroporate single, adherent, tissue-cultured cells while others have used microfabricated electrodes. [26-29]. Several of the above designs successfully employed single, brief (about 1 ms) DC voltage pulses to perform electroporation.

If the applied transmembrane voltage exceeds a critical value, pore formation becomes irreversible and cell lysis occurs. Some investigators have used this observation to move from electroporation to electrical lysis of cells. In one method, Cheng and coworkers reported the use of a pulsed, high voltage field of 20 second duration to lyse cells on a microfabricated device for a variety of applications including obtaining DNA and RNA from bacteria and the destruction of unwanted cells. [19, 20]. These studies showed that combined AC fields for the study of membrane deformation or for dielectrophoretic separation of blood cells or E. coli cells, could also be used for cell lysis over a range of frequencies from 20 KHz to 1 MHz with potential differences of 40 to 2 kV/cm respectively. Krueger's studies of membrane deformation by electrical fields in the context of electroporation also reported cellular lysis with field strengths in this range. [25]. Cheng et al were able to achieve lysis of E. coli with several hundred bursts of an AC field of 500 volts each of 50 microseconds duration across an array o electrodes on a substrate with an inter electrode gap of about 200 microns. [19].

Similarly, Lee and coworkers describe a micro-machined cell lysis device based on a similar theory. [21]. Ramsey and colleagues also utilized AC electrical fields for fast, high throughput lysis of cells on a microfluidic device followed by separation of the intracellular contents. [see 22 and U.S. Pat. Publication No. 20030075446]. Yeung's group lysed a lymphocyte preloaded into a capillary with a 15-20 second exposure to an electric field from a Tesla coil. Lysis of the cell required 15-20 seconds by this method and the cell was also required to stick to the inner surface of the capillary. [10].

In another area of endeavor, other investigators have prepared surfaces of substrates with patterned electrodes [41, 42]. Standard vapor deposition methods in the presence of a mask were used to pattern the surface of a coverslip with electrodes composed of indium tin oxide (which is transparent) or an alternative metal. Other investigators have prepared other surface molecules for cell attachment. [43].

While the foregoing studies have made significant contributions to the analysis of cells, subsecond electrical lysis of single adherent cells followed by rapid loading of the cellular constituents into a micro-collection device such as a capillary electrophoresis tube for separation has yet to be demonstrated. Moreover, all of the prior art methods for adherent cells require several seconds and/or several pulses to disrupt cell membranes and therefore are generally appropriate only for investigation of the properties of static or slowly changing cellular analytes, such as DNA, RNA, or protein content. Many biochemical reactions occur on time scales of seconds or less, thus the temporal resolution (i.e. the time to terminate cellular reactions) of the sampling technique becomes critical when analyzing cellular properties such as enzymatic activity. For such biochemical analyses, accurate measurement requires complete cell lysis with termination of biochemical reactions in subsecond time periods. For non-adherent cells, in a system described by Ramsey, U.S. Pat. Pub. No. 2003007544, subsecond lysis could be achieved when the cell was loaded into a microfluidic device. A drawback of such a microfluidic device, as with the internal capillary lysis system described by Xue and Yeung [10] is that the cell must be drawn within the interior of the capillary or microfluidic device, which disturbs the resting state and resulting biological activity of the cell, potentially leading to artifacts.

Finding an inexpensive and easy-to-use alternative for rapid cell lysis would be beneficial for the analysis of rapid cellular processes. What is needed in the art are new apparatus and methods for rapid cell lysis that maintains the subsecond temporal resolution and optical compatibility of laser-mediated lysis, but overcomes the limitations of cost and difficulty of use. Moreover, what is needed is a method to lyse a single selected cells with a single subsecond pulse of an electric field and to collect the contents thereof substantially simultaneously with the lysis of the cell.

### SUMMARY OF THE INVENTION

The invention is directed apparatus and methods to rapidly lyse a biological cell using a single electrical pulse of subsecond duration followed by efficient loading of the cellular contents into a micro-collection device. Micro fabricated electrodes are described that create a maximum voltage drop across a cell's plasma membrane at a minimum inter-electrode voltage. The influence of the inter-electrode distance, pulse duration, and pulse strength on the rate of cell lysis are described. The ability to rapidly lyse a cell and collect and separate the cellular contents is demonstrated by loading cells with the exemplary flourophores Oregon Green and two isomers of carboxyfluorescein. All three fluorophores are detected from lysed with a separation efficiency comparable to that of standards.

In one aspect, the invention provides embodiments of cell chambers configured with electrodes and surface materials for electrical lysis of cells. In one embodiment, the cell chamber includes a substrate material having a bottom and a top surface. An electrode is layered on the top surface of the substrate material; and a cell adhesion material is layered on top of the electrode. In certain embodiments, the substrate material and the electrode material are transparent materials. In certain embodiments, the cell adhesion material is a hydrophilic region layered over the electrodes and the area between the electrodes is layered with a hydrophobic region. Together the hydrophobic and hydrophilic regions pattern the cells on the cell chamber over the electrodes. In another embodiment, the cell chamber includes an array of cell adhesion materials comprised of a plurality of hydrophilic regions above a plurality of electrodes in an array with the plurality of hydrophilic regions being separated from one another by adjacent hydrophobic regions.

In another embodiment, the cell chamber includes a substrate material for receiving a plurality of cells, the substrate material having a top surface and a bottom surface. The substrate includes an array of openings through the substrate material between the top surface and the bottom surface. The openings provide a conductive pathway for electrical current to flow between electrodes placed above and below the openings.

In another embodiment, the cell chamber includes an array of separately addressable electrode pairs on the top surface of the substrate, each of the electrode pairs comprising a first electrode and a second electrode of opposite polarity. The first and second electrodes are separated from one another by a distance of less than 200 microns, and the array of electrode pairs are separated from each other by a distance greater than 200 microns. The cell chamber also includes an array of cell adhesion materials comprised of a plurality of hydrophilic regions separated from one another by adjacent hydrophobic regions and positioned above the array of electrode pairs so that the plurality of hydrophilic regions are located above the plurality of electrode pairs.

In another aspect, the invention provides micro-collection devices for electrical lysis of a biological cell and collection of at least a portion of contents thereof. In one embodiment, the micro-collection device includes a micro capillary electrophoresis tube having an exterior wall encircling an interior lumen, a distal end for contacting an electrolyte and a proximal end for collecting at least a portion of the contents of a lysed cell. The proximal end preferably has an outer diameter of less than about 200 microns. The capillary electrophoresis tube has and an electrode pair attached to the proximal end where the electrode pair includes a first and a second electrode separated from one another by distance of less than 200 microns.

In another embodiment, the capillary electrophoresis tube has an exterior wall encircling an interior lumen, the exterior wall having a thickness of less than 200 microns. The exterior wall has a pair of electrodes including a first electrode on an exterior surface thereof and a second electrode on an interior surface thereof so that the electrodes are separated from one another by a distance of less than 200 microns. The electrode pair is attached to the proximal end of the capillary electrophoresis tube.

Another embodiment includes the capillary electrophoresis tube having an exterior wall encircling an interior lumen, a distal end for contacting an electrolyte and a proximal end for collecting at least a portion of the contents of a lysed cell The proximal end is tapered and has an outer diameter of less than 200 microns. The capillary electrophoresis tube further includes an electrically conductive material deposited on the proximal end of the tube and a conductive lead extending down a portion of the length of the tube contacting the electrically conductive material.

In another embodiment, the capillary electrophoresis tube includes a removably insertable wire within the lumen of the capillary electrophoresis tube at a distal end thereof opposite the proximal end that collects the sample. When the wire is inserted into the lumen at the distal end in the presence of an electrolyte, the wire is put into electrically conductive contact with the electrolyte, making the electrolyte in the lumen serve as a liquid electrode.

In another aspect, the invention includes systems that use one or more of the foregoing cell chambers or capillary electrophoresis tubes. The system includes electrical means to lyse the biological cell within 1 second or less of application of an electrical potential to the electrical means and a micro-collection means configured to collect at least a portion of the contents of the lysed cell within a period of about 1 second or less from lysing the cell. In certain embodiments the system commences collection simultaneously with lysis.

One embodiment of such a system includes a first electrode that is at least one of positioned on a substrate or positionable within less than 200 microns from a biological cell in contact with the substrate. It also includes a second electrode that is at least one of positioned on the substrate less than 200 microns of the first electrode or positionable less than 200 microns of the first electrode. The first and second electrodes are thereby configured for positioning a biological cell there between. The system further includes a micro-collection device having a proximal end configured to capture at least a portion of the contents of the biological cell and being at least one of positioned or positionable less than 200 microns of at least one of the first and second electrodes. The micro-collection device is configured to collect at least the portion of the contents of the biological cell within less than about 1 second of lysis, but the micro-collection device is not configured to hold the biological cell or to be in contact with the biological cell during lysis.

In another aspect, the invention provides methods of electrical lysis of a biological cell and collection of at least a portion of the contents thereof, that include the acts of depositing the biological cell on a substrate, providing electrical means to lyse the biological cell on the substrate within 1 second or less of application of an electrical potential to the electrical means; and collecting at least a portion of the contents of the lysed cell with a micro-collection means configured to collect the contents of the lysed cell within a period of about 1 second or less from lysing the cell.

In various embodiments of the methods, a single subsecond pulse of an AC or DC field is applied across the biological cell using the forgoing devices of the invention. In certain embodiments, the act of collecting is commenced simultaneously with the act of electrical lysis. In other embodiments, the act of collecting commences within 1 second, within 100 ms, within 10 ms, within 1 ms, within 100 μs within 10 μs or within 1 μs of commencing the electrical lysis. Electrical lysis is commenced by applying an electrical potential of about 5 to about 30 Kv/cm in one embodiment, about 10 to 20 Kv/cm in another embodiment, or about 15 Kv/cm in another embodiment, between the first electrode 18 and the second electrode 19. The electrical potential is applied for a duration of about 10 ms or less in certain embodiments, or 1 ms or less, or 100 μs , or 10 μs or less in other embodiments.

### BRIEF DESCRIPTION OF THE DRAWINGS

FIG. 1 depicts a top view of a cell chamber with an array of first electrodes for electrical lysis of a biological cell according to one embodiment of the invention.

FIG. 2 depicts a side view of a cell chamber with an array of first electrodes for electrical lysis of a biological cell in combination with a capillary electrophoresis tube configured with a second electrode on the proximal end.

FIG. 3 is a top view of an embodiment of a cell chamber configured with independently addressable electrodes.

FIG. 4 is a top view of another embodiment of a cell chamber configured with independently controllable electrodes.

FIG. 5 is a flow diagram that depicts a method of making a cell chamber with first electrodes having hydrophilic regions on top separated by hydrophobic regions between the electrodes.

FIG. 6 is a top view of a cell chamber configured with electrode pairs according to another embodiment of the invention.

FIG. 7 is a side view of another embodiment of a cell chamber and capillary electrophoresis tube with an electrode on the exterior of the proximal end of the tube in an embodiment of the invention.

FIG. 8 is a side view of is a side view of a cell chamber with an array of first electrodes and a capillary electrophoresis tube with a wire in the distal end of the lumen according to another embodiment of the invention.

FIG. 9 is a side view of a cell chamber with an array of first electrodes and capillary electrophoresis tube with the second electrode and a tapered proximal end according to another embodiment of the invention.

FIG. 10 is a side view of a cell chamber with a single first electrode and capillary electrophoresis tube with the second electrode and a tapered proximal end according to another embodiment of the invention

FIG. 11 is a side view of a cell chamber without electrodes and one embodiment of a capillary electrophoresis tube configured with both first and second electrodes on the proximal end according to another aspect of the invention.

FIG. 12 is a side view of a cell chamber without electrodes and a capillary electrophoresis tube configured with first and second electrodes on the proximal end according to another aspect of the invention.

FIG. 13 are side views of a biological chip micro-collection device in other embodiments of the invention. FIG. 13A depicts a biological chip useful with a cell chamber having an electrode pair according to the invention. FIG. 13B depicts the biological chip configured with a second electrode useful with cell chambers having a first electrode according to the invention. FIG. 13C depicts the biological chip configured with first and second electrodes.

FIG. 14 are side views of an optical fiber bundle micro-collection device in other embodiments of the invention. FIG. 14A depicts the optical fiber bundle useful with a cell chamber having an electrode pair according to the invention. FIG. 14B depicts the optical fiber bundle configured with a second electrode useful with cell chambers having a first electrode according to the invention. FIG. 14C depicts the optical fiber bundle configured with first and second electrodes.

FIG. 15 depicts a system of the invention that includes a micro delivery device in conjunction with the capillary electrophoresis tube micro-collection device.

FIG. 16 depicts two different placements of electrodes and fields for electrical lysis according to methods of the invention.

FIG. 17 are graphs depicting cellular lysis times as a function of electrical pulse height (17A), pulse width (17B) and distance between electrodes (17C) in a method of the invention.

FIG. 18 are photographs depicting electrical lysis of cells according to the methods of the invention.

FIG. 19 shows electrophoresis charts depicting the separation of flourophores from electrically lysed cells according to methods of the invention.

FIG. 20 is a chart depicting similar collection efficiency of the electrical methods of the present invention in comparison to laser based methods of the prior art.

FIG. 21 illustrates an array of capillary electrophoresis tubes with electrodes in combination with an array of first electrodes according to another embodiment of the invention.

### DETAILED DESCRIPTION OF PREFERRED EMBODIMENTS

Prior to setting forth the present invention in detail, the meanings of certain terms used herein are provided to aid in an understand thereof. These definitions are not intended to be exclusive, but rather are inclusive of the ordinary meanings of the terms, except if the ordinary meaning is construed to conflict with definitions set forth below.

“Attached” with respect to an electrode or conductive material in reference to a component part means that the electrode or conductive material is firmly in contact with the component part. In one embodiment “attached” means that a layer of the conductive material is deposited on the component part directly or indirectly through another layer in contact with the component part. In another embodiment “attached” means that the conductive material is bonded to the component part though an adhesive, weld or other bonding means. In another embodiment “attached” means that the conductive material is fixed or fastened to the component part through a mechanical contact.

“Transparent” means that a substrate, coating or layer of material permits transmission of at least 10 percent of incident light having a wavelength in the ultraviolet or visible spectrum.

A “micro-collection device” is a device configured to collect small amounts of material from a lysed cell in a small volume. The material is collected in a volume that is less than about 10 nanoliters, less than about 1 nanoliters, less than about 100 picoliters, less than 10 picoliters or less than 1 picoliter. In typical embodiments, the material is collected from a volume of about 0.5 picoliter to about 1 nanoliter of volume.

A “capillary electrophoresis tube” is a micro-capillary configured on a proximal end to collect a small amount of sample material and on a distal end to be in contact with an electrophoresis buffer. In typical configurations, the capillary electrophoresis tube contains at least one window through the tube permitting analysis of sample material that is at least partially separated into zones as a result of electrophoretic and/or electo-osmotic movement in the tube. In certain embodiments, the capillary electrophoresis tube may include a gel in the interior lumen of the tube to facilitate the electrophoretic separation. Capillary electrophoresis tubes are distinguishable from ordinary micro capillaries, typically by having a length of 20 cm or greater and/or by having outer walls surrounding the lumen where the outer walls are covered by a protective coating such as polyimide.

A “cell chamber” means any substrate material having a surface upon which a sample including a biological cell can be applied. The term includes, but is not limited to, conventional microscope cover slips, conventional microscope glass slides, reaction vessels, tissue culture containers, dishes and the like. The cell container is preferably in a configuration that can be adapted for mounting on an instrument for detecting the position of the cell located in or on the container.

A “cell adhesion material” or “cell adhesion layer” is an electrically permeable biologically non-toxic material that adheres to a biological cell, specifically or non-specifically, and that is deposited on a surface above an electrode at a density that permits electrical conductivity from the electrode through the deposited material into an electrolyte above or below the cell adhesion materials. Thus, a cell adhesion material does not include ordinary solid substrate materials such as glass, polystyrene or polypropylene on which cells may adhere but that does not permit the passage of current. Example non-specific cell adhesion materials include hydrophilic molecules such as those containing anionic moieties or cationic moieties in the presence of the electrolyte at a selected pH. Polylysine is one example of a non-specific hydrophilic molecule suitable for a cell adhesion layer. Examples of specific cell adhesion materials include proteins, particularly antibodies or fragments thereof, lectins, carbohydrates, fibronectin, collagen, laminins, vitronectin, endothelial cell attachment factor, integrin ligands, Matrigel, endothelial cell attachment factor, nucleic acids, ligands that bind molecules on the surface of cells or other members of a biological binding pair.

Throughout the description that follows, citation is made to various references that may aid in understanding or practice of various embodiments of the invention. Each such reference is herein incorporated by reference in its entirety to the extent their teachings do not conflict with the requirements of the invention described herein.

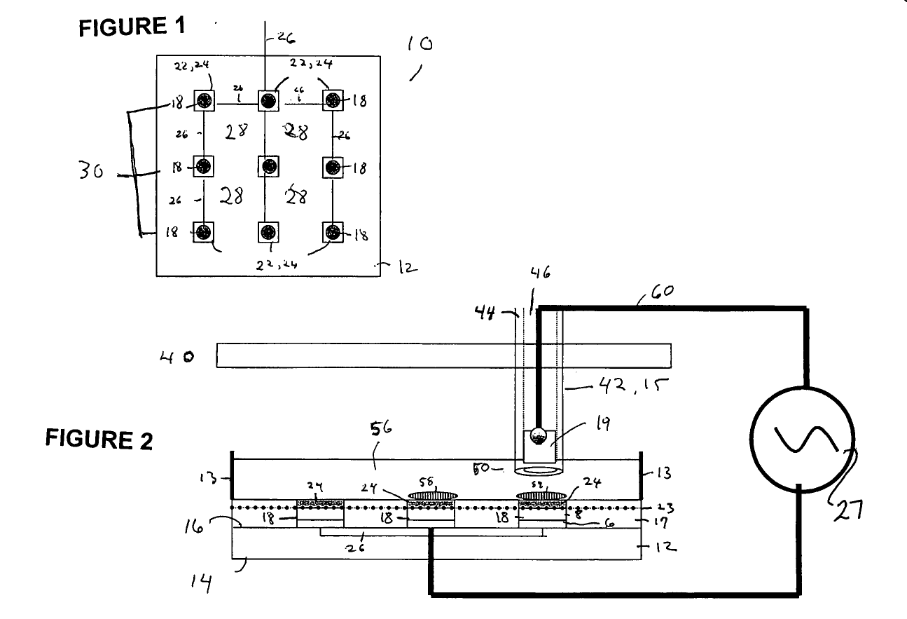

Turning now to the description of exemplary embodiments of the invention, one aspect of the invention includes various embodiments of cell chambers 10 configured to facilitate rapid electrical lysis of a biological cell. FIGS. 1-4 and 6-10 show various examples of the inventive cell chambers 10 alone or in combination with micro-collection devices 15 (exemplified by capillary electrophoresis tubes 42) used in various configurations of the invention. A common feature of the various embodiments of the invention, is that to implement electrical lysis of cells, electrodes of 2 different polarities (ground and source in a DC configuration/alternating positive and negative in the AC configuration).

FIGS. 1 and 2 illustrate top and side views, respectively, of one embodiment of a cell chamber 10 of the invention. The cell chamber 10 includes a lower substrate material 12 that in certain embodiments, is a transparent material such as in a cover slip or glass slide. Suitable materials for the lower substrate material 12 include, but are not limited to glass, quartz, silicon nitride or a transparent plastic such as polydimethyl siloxane (PDMS), polystyene, polycarbonate, polyethylene, polymethyl methacrylate (PMMA) and the like. The substrate material 12 may be configured to fit onto a suitable instrument for detecting the position of biological cells 58 and 59 in the cell chamber 10. The substrate 12 has a top surface 16 and a bottom surface 14. A fist first electrode 18 is located on the top surface 16. The first electrode 18 may be a transparent electrode in certain embodiments. A transparent electrode may be made for example, of indium tin oxide or a layer of one or more conductive metal less than about 300 angstroms in thickness. For a traditional metal such as gold 200-300 angstroms is the approximate upper limit of transparency. For a metal such as indium tin oxide (ITO) thickness greater than 1000 angstroms can be used with retention of transparent properties. The first electrode 18 may be a single metal, an alloy, or composite structure. In the exemplary embodiment illustrated in FIG. 1, the first electrode 18 is a composite of layers of metal that includes a lower layer 6 of titanium of about 50 angstroms thickness deposited on the top surface 16 of the substrate material 12 and an upper layer 8 of gold of about 80 angstroms in thickness deposited over the lower layer 6 of titanium.

In the embodiment illustrated in FIG. 1, the first electrode 18 is one of a plurality of electrodes 18 arranged in an array 30 on the top surface 16 of the substrate material 12. The first electrode 18 is connected to a conductive lead wire 26 configured to put the first electrode 18 in conductive contact with ground or one pole of an electrical power supply 27. In certain embodiments, the electrical power supply 27 can be a DC source while in other embodiments, the electrical power supply 27 is an AC source. AC power sources suitable for the invention provide alternating current at a frequency greater than 1 kilohertz. In certain embodiments the frequency is greater than 10 kilohertz, In other embodiments the frequency is greater than 100 kilohertz. In still other embodiments the frequency is one megahertz or greater.

FIG. 2 shows a side view of the cell chamber 10 of FIG. 1 as used in a system in combination with a micro-collection device 15 (capillary electrophoresis tube 42) configured with a second electrode 19 on a proximal end 50 thereof. The capillary electrophoresis tube 42 depicted in FIG. 2 includes an exterior wall 44 enclosing an interior lumen 46 in which the contents of lysed cell will be collected and separated by electrophoresis. The system is preferably configured with a selectable positioning device 40 to move at least one of the cell chamber 10 or the capillary electrophoresis tube 42 relative to one another, so as to place the proximal end 50 of the capillary electrophoresis tube 42 at a selectable position above a selected cell 59 and simultaneously place the second electrode 19 less than about 200 microns, less than about 100 microns, less than about 60 microns, or 20 microns or less from the first electrode 18 located beneath or adjacent to the selected cell 59.

The array 30 of electrodes 18 are separated from one another by a non-conductive layer 17 of material that is preferably also a transparent material. The thickness of the non-conductive layer 17 may be a varied according to particular applications and/or manufacturing constraints. Any thickness suitable for the observation or detection instrument may be used. In advantageous embodiments, the non-conductive layer 17 is about 10 to 10,000 angstroms in thickness, in other embodiments the non-conductive layer 17 is about 50 to 1000 angstroms, in preferred embodiments about 100-500 angstroms and in typical embodiments about 200 angstroms in thickness. Suitable materials for the non-conductive layer 17 include but are not limited to, air, glass, quartz, silicon nitride, or a transparent plastic such as PDMS, silicon nitride, polystyene, polycarbonate, polyethylene or other non-conductive substance that is transparent at the thickness applied. Preferably the non-conductive layer 17 is made of material such as PDMS or silicon nitride that has silanol-reactive chemical moieties that can be used to pattern the upper surface of the non-conductive layer 17 with different types of materials, either directly, or indirectly through a binding layer 23. The binding layer 23 may be made of any suitable material for cross linking to the different types of material used to pattern the surface of the uppermost surface of the cell chamber 10. The binding layer 23 should preferably be deposited at a sufficiently low density to provide electrical conductivity between the first electrode 18 and the electrolyte medium 56.

The different materials used to pattern the surface include at least one cell adhesion material 22 deposited above the first electrode 18 and one hydrophobic material 28a deposited away form the first electrode and/or between electrodes 18 in the array 30 The hydrophobic material 28a forms hydrophobic regions 28. In non-specific embodiments, the cell adhesion material 22 is a hydrophilic material 24a. The hydrophilic material 24a located above the first electrode a forms a hydrophilic region 24 that is a functional part of cell adhesion layer 22. The hydrophilic regions 24 facilitates adhesion of a biological cell 58 to the cell chamber 10 above the first electrode 18. The hydrophobic regions 28 also facilitate patterning of the biological cells 58, 59 on the cell chamber 10 above the electrodes 18 because biological cells 58, 59 do not typically adhere to the hydrophobic regions 28 but tend to adhere very well to the hydrophilic regions 24. Suitable materials for forming the hydrophilic regions 24 include, but are not limited to, poly amines such polylysine, self assembled monolyaers with polar or charged end groups, palladium, gold, glass, metal oxides, poly(N-isopropylacrylamide), palladium, gold, metal oxides, porous glass or other cell adhesion material, Suitable materials for forming the hydrophobic regions 28 include, but are not limited to, polyethylene glycol (PEG) monomers, self assembled monolayers with terminal carbon chains of C4 or greater in length, cellulose acetate, paraffin, agarose, sulfonate-terminated alkylsilanes, PEG-derivatized alkanethiol SAMs, interpenetrated copolymers of PEG, alkanethiol SAMs functionalized with tri(propylene sulfoxide) fluorocarbon polymers, poly(vinyl alcohol) multilayers of PEG functionalized with silicon chloride, polyethylene oxide, and the like.

The positioning of the second electrode 19 in close proximity to the first electrode 18 provides for separate addressing of each electrode 18 in the array. When the electrodes 18 in the array 30 are linked to a common contact lead 26 as illustrated in FIG. 1, each of the electrodes 18 in the array 30, including those not specifically addressed by placing the second electrode 19 immediately above, will generate some unwanted amount of angular electrical field potential with the second electrode 19. To minimize these angular field effects with non-addressed electrodes, each of the electrodes 18 in the array 30 should preferably be separated from each other by a distance greater than 200 microns in certain embodiments, greater than 300 microns in other embodiments, or greater than 500 microns in other embodiments.

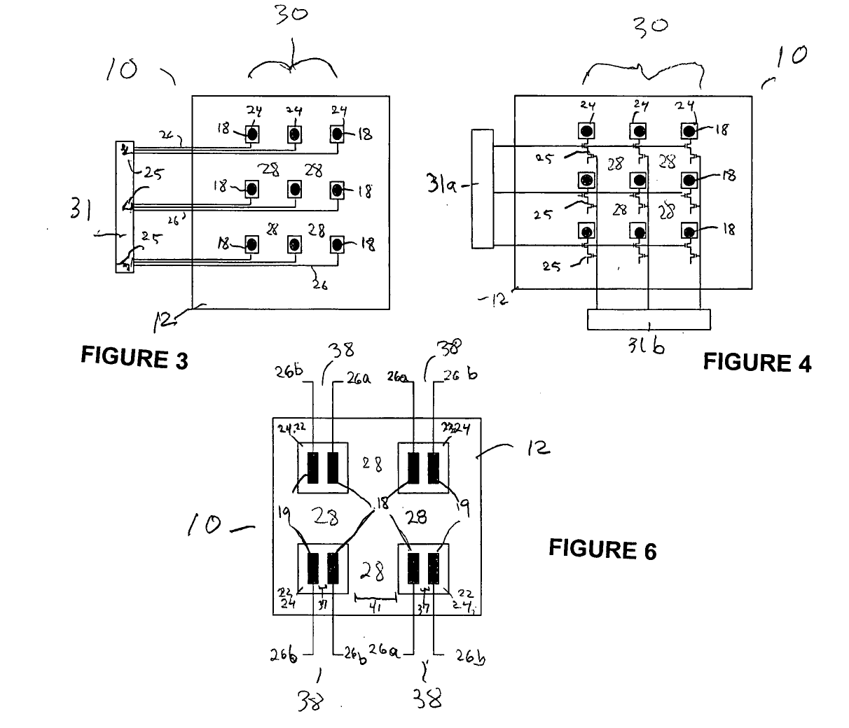

Another way to minimize unwanted field effects is to have each electrode 18 in the array of electrodes independently connected to independently controllable switches 25 as illustrated in FIG. 3 and 4. These switches 25 may be independently controlled by switch control module 31. FIG. 3 illustrates a single switch control module 31 that separately controls each individual electrode 18 using separate switches within the control module. FIG. 4 illustrates a switch control module 31, that uses row 31a and column 31b addressing modules that activate selected rows and columns of the controllable switches 25 in the array so that only one electrode 18, having both the row and the column address switches 25 activated will be placed in conductive contact with ground (or one pole of the power source 27). FIG. 4 shows the switch elements 25 being located on the cell chamber 10 for illustrative purposes. In certain embodiments, standard lithographic techniques can be used to fabricate appropriate controllable switch elements 25 directly on the cell chamber 10 beneath the electrodes 18. In other embodiments, however, the switch elements 25 may be located within the control module 31a, 31b requiring only separate conductive leads 26 to each of the electrodes 18 as depicted in FIG. 3.

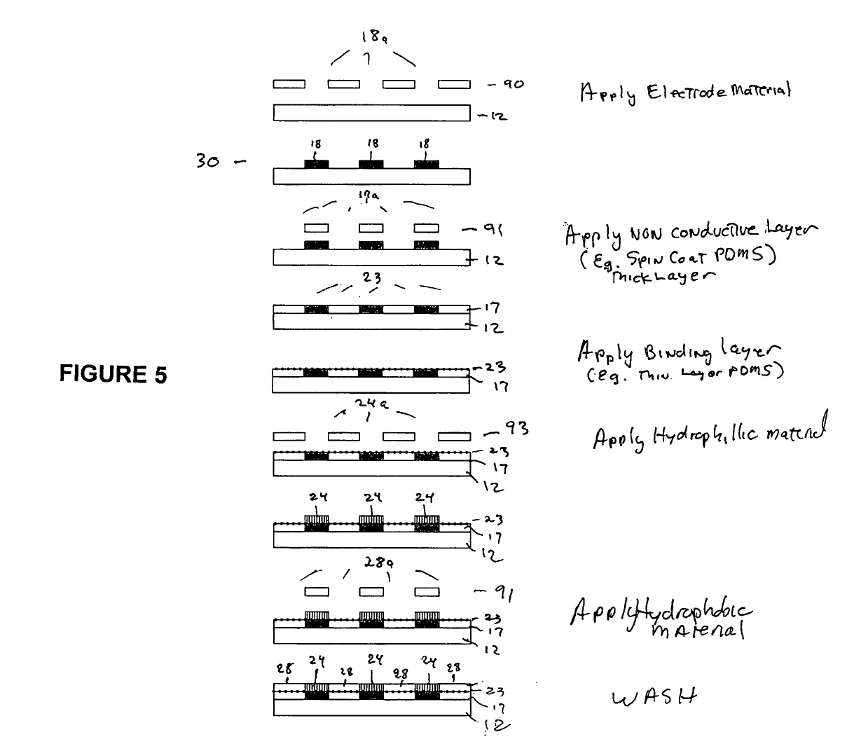

FIG. 5 illustrates an example method of forming a patterned layer of hydrophobic regions 28 adjacent of hydrophilic regions 24 in a plane on the cell chamber 10 where the hydrophilic regions are above electrodes 18. A first overlay mask 90 is positioned above the substrate material 12 and a layer of electrode material 18a is deposited on the substrate through the first overlay mask 90 forming the array 30 of electrodes 18 on the top surface 16 of the substrate material 12. A second overlay mask 91 is configured to block the surface of the electrodes 18 and the non-conductive layer 17 is deposited on the substrate 12 through the second mask 91 using a suitable process for the particular non-conductive material used for the layer, for example, by spin coating when the non conductive material 17 is PDMS. If the non-conductive material is PDMS or similar substance that is insulating if in a dense layer but can allow a current to be conducted when applied in a thin layer, the substance is first applied as a lower layer 3 having a density sufficient to provide electrical insulation between the electrodes 18. Then it is applied as an upper layer 5 at a lower density sufficient to provide pores so that the PDMS layer will provide electrical conductivity between the electrode 18 and the electrolyte medium 56. In that case, the upper layer of PDMS or other substance also functions as the binding layer 23 although being made of the same material as the non-conductive layer 17.and the second mask 91 may be removed prior to depositing the upper layer so that the upper layer 5 is also deposited on the electrodes 18. In an alternative process not illustrated, a separate binding layer 23 is applied over the non-conductive layer 17. The non-conduct layer 17 is cured (or otherwise fixed) onto the substrate 12 in a curing step 88. A third overlay mask 93 having a pattern corresponding to the first overlay mask is positioned over the cell chamber 10 and the hydrophilic material 24a is adsorbed or otherwise linked to the cell chamber 10 by being deposited through the third mask 93. The third mask 93 is removed and the cell chamber 10 is washed, thereby establishing the hydrophilic regions 24 positioned above the electrodes 18. The hydrophobic material 28a is then deposited over the exposed non-conductive layer 17 by a suitable process such as grafting when the hydrophobic material 28a is a polyethylene glycol monomer using a suitable process, for example as described in Hu et al, [44]. Other suitable methods for manufacturing the patterned surface of cell adhesion materials 22 and hydrophobic regions 28 include, but are not limited to methods described by Folch et al, Kane et al, Craighead et al, Jung et al, and Whitesides et al. [43, 45-48].

Although not illustrated in FIG. 2, it will be understood that similar masking techniques can be used to lay down the conductive contacts 26 to the electrodes 18.

In this exemplary process, where the hydrophilic material 24a is polylysine deposited above the electrode and the hydrophobic material 28a is PEG, the formation of the hydrophilic region 24 blocks PEG from being grafted to the hydrophilic regions. It should be understood, however, that where a selection of different materials for the cell adhesion layer 22, the hydrophilic region 24 and/or the hydrophobic regions 28 is used, other physical or chemical processes suitable for the selected materials are used to fabricate the layers. Moreover, other suitable lithographic masks may additionally be employed to protect from unwanted depositions of the hydrophobic material 28a in the same vicinity as the hydrophilic regions 24.

The process illustrated in FIG. 5 is for exemplary purposes only. A variety of lithographic methods may be used to pattern the cell chambers 10 described in the present invention. Further suitable process for example, include those described by Hu et al, [44] or by Chen et al., [421 or by Cheng et al, [19 20], in combination with methods described by Folch et al, Kane et al, Craighead et al, Jung et al, and Whitesides et al. [43, 45-48].

FIG. 6 illustrates another embodiment of a cell chamber 10 according to the invention. In this embodiment, electrode pairs 38 are deposited on the substrate 12 rather than a single electrode or array of electrodes. The electrode pairs 38 have both the first electrode 18 and the second electrode 19 of opposing polarity located on the substrate 12. The first electrode 18 and the second electrode 19 are separated from one another by first distance 37 that is less than about 200 microns in one embodiment, or less than 100 microns, or less than 60 microns, or less than 40 microns, or about 20 microns or less in various other embodiments. Each of the electrode pairs 38 has contact leads 26a and 26b to place the electrodes in conductive contact with opposite poles of power supply 27 and/or to ground. As with the other embodiments of the invention, this first distance 37 is sufficiently close to lyse a cell positioned between the first 18 and second 19 electrodes within less than one second in certain embodiments, within less than 300 ms in other embodiments, or within 33 ms or less in still other embodiments. Lysis occurs when an electrical potential of about 5 to about 30 Kv/cm in one embodiment, about 10 to 20 Kv/cm in another embodiment, or about 15 Kv/cm in another embodiment, is applied across the pair of electrodes 38 in a single pulse of a duration of about 10 ms or less, 1 ms or less, 100 microseconds or less, or 10 microseconds or less in various embodiments.

The electrode pairs 38 are separated from one another by a second distance 41, greater than the first distance 37, where the second distance 41 is sufficiently far to prevent electrical lysis of a cell located between pair of electrodes 38b, 38c, and 38c when the difference of potential of about 10 to about 50 volts is applied across a selected set of electrode pairs 38a. In various embodiments, the second distance 41 is greater than 200 microns, greater than 300 microns, or in other embodiments, greater than 500 microns. A layer of cell adhesion material 22 is deposited over the electrode pairs 38 and a hydrophobic region 28 is deposited over the substrate material adjacent and between the electrode pairs 38. The cell adhesion material 22 again may be formed of hydrophilic regions 24 located over the electrode pairs 38. The lead wires 26a and 26b are used place the first 18 and second 19 electrodes of the electrode pair 38 in conductive contact with an electrical power supply or ground. In certain embodiments, the electrodes 18 and 19 of the electrode pair 38 may be transparent electrodes, however, if the substrate 12 and/or non-conductive layer 17 below the electrode pair 38 is transparent, it is not necessary to also make the electrodes 18 and 19 transparent because a cell deposited between the electrode pairs 38 will be visible through a microscope or other suitable optical viewing instrument.

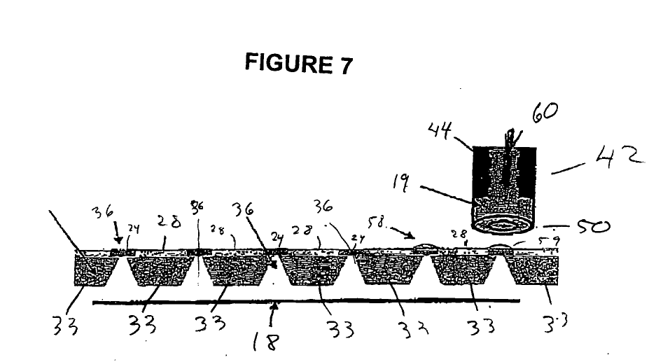

FIG. 7 illustrates another embodiment of a cell chamber 10 of the invention. In this embodiment no electrodes 18 or electrode pairs 38 are deposited on the substrate 12. Rather, the substrate material 12 is configured with a plurality of conductive openings 36 between the top surface 16 and the bottom surface 14 of the substrate material 12. The conductive openings 36 are separated from one another by a plurality of adjacent non-conductive regions 33. The size of the conductive openings is 36 is selected to be smaller than the size of the biological cell 58 to be lysed. The top surface 16 of the substrate material 12 is coated with a plurality of hydrophobic regions 28 deposited above the plurality of non-conductive regions 33. The conductive openings 36 may optionally include hydrophilic materials 24 deposited over a porous mesh of the substrate material 12. In these embodiments, the cells 58, 59 deposited on the top surface 16 of the cell chamber 10 will preferentially locate above the conductive openings 36. The cell chamber 10 is located above a metallic layer or wire that forms the first electrode 18. In certain embodiments, the metallic wire is separated from the bottom surface 14 and/or the conductive openings by a volume into which the electrolyte 56 is also contained below the bottom surface.

While the embodiments of cell containers 10 described herein before each include transparent materials for the substrates layer 12, and/or the non-conductive layer 17, and/or the electrodes 18 and/or the hydrophobic 28 and hydrophilic 24 layers, such transparent materials are only for preferred embodiments for use with a transmissive optical viewing instrument such as a microscope. The invention is not, however, limited to the use of such transparent materials for any of the structures described herein. In other embodiments, these structures may be made of opaque rather than transparent materials, especially when configured for use with reflective microscopes or other detection instruments that do not rely on the transmission of light through the cell containers 10.

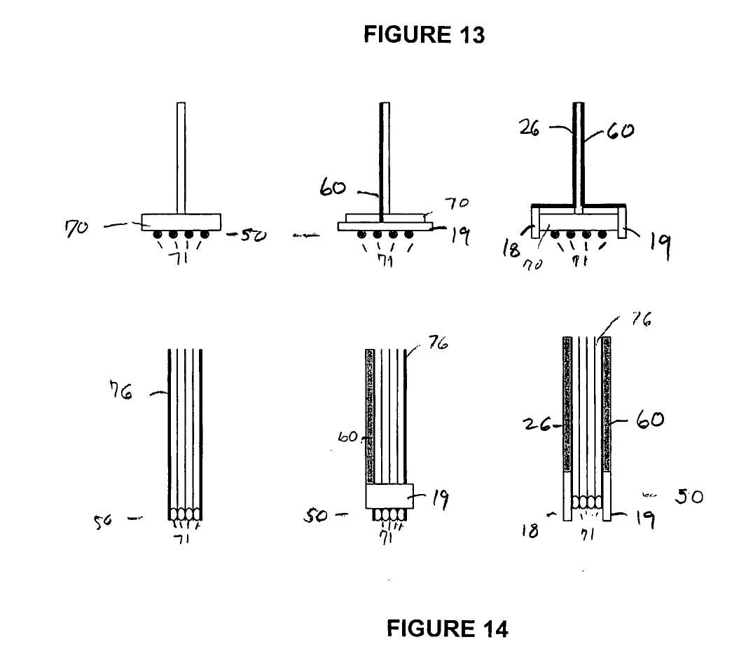

Another aspect of the invention illustrated in FIGS. 2 and 7-12, is system used with the various cell chambers 10 of the invention, which includes a micro-collection device 15 (i.e., capillary electrophoresis tube 42) configured with one or more electrodes to facilitate both rapid lysis of a single selected cell and collection of at least a portion of the contents thereof all within one second or less The micro-collection devices 15 illustrated in these Figures are capillary electrophoresis tubes 42. Other exemplary embodiments of a micro-collection devices 15 are illustrated in FIGS. 13 and 14.

The capillary electrophoresis tubes 42 of FIGS. 2 and 7-10 are configured with the second electrode 19 at the proximal end 50 thereof. The second electrode 19 has a second electrical lead 60 to put the second electrode 19 in contact with the power supply 27 (or ground source) having a polarity opposite the polarity of the first electrode 18 located on the cell chamber 10. At least one of the cell chamber 10 and the capillary electrophoresis tube 42 is moveable relative to one another to selectively position the second electrode 19 over a selected cell 59 and above the first electrode 18. An O-ring, gasket, or other barrier 13 that extends above the upper surface of the cell chamber 10 and surround a perimeter provides a reservoir for containing a conductive medium 56 such as a buffer or electrolyte. The conductive medium 56 is placed between the first electrode 18 and the second electrode 19 so that the proximal end 50 of the capillary electrophoresis tube 42 with the second electrode 19 attached thereto is positioned over, or in proximity to, the selected biological cell 59 by a distance of less than about 200 microns in one embodiment, or less than 100 microns, or less than 60 microns, or less than 40 microns, or about 20 microns or less in various other embodiments. A single pulse of electrical potential of less than one second is applied between the first 18 and second electrodes 19 so that electrical current flows between the first electrode 18 and the second electrode 19 causing lysis of the selected cell 59 within less than one second.

In the embodiment illustrated in FIG. 7, the electrical current flows between the lower electrode 18 via the conductive openings 36, through the selected cell 59 and electrolyte medium 56. In a modification of the embodiment shown in FIG. 7, at least one of the cell chamber 10 and the first electrode 18 are moveable relative to one another to position the first electrode 18 beneath a selected conductive opening 36 under a selected cell 59 while the position of the second electrode 19 on the capillary electrophoresis tube 42 remains fixed. This embodiment minimizes angular field potential between the first lysing electrode 18 and other conductive openings 36 not positioned directly beneath the second lysing electrode 19 on the capillary electrophoresis tube 42 located directly above the selected cell 59.

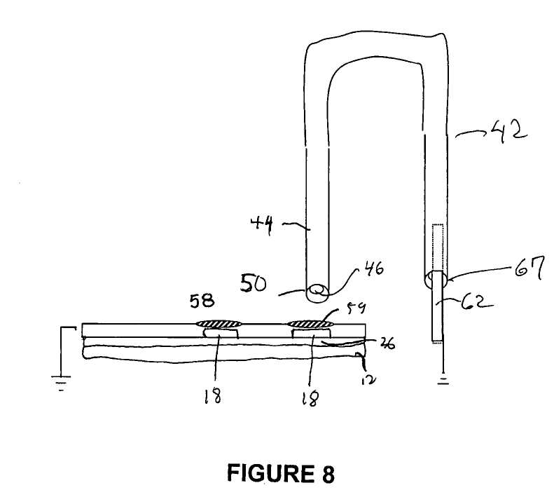

FIG. 8 illustrates another embodiment of a capillary electrophoresis tube 42 configured with an electrode 19 at the proximal end. The capillary electrophoresis tube 42 is configured with a lead wire 62 at the distal end 67. The lead wire 62 is connected to one pole of the power supply 27 at one end, and is in electrically conductive contact with the electrolyte material 56 within the lumen 46 of the capillary electrophoresis tube 42 at the other end. In this embodiment, the electrolyte material 56 within the lumen functions as the second electrode 19 in liquid form, so that when the lead wire 62 is charged with a different of potential with the first electrode 18, current flows between the first electrode 18, the electrolyte medium 56 within the lumen, and the lead wire 62 at the distal end causing electrical lysis of the selected cell 59 positioned beneath the proximal end 50 of the capillary electrophoresis tube 42.

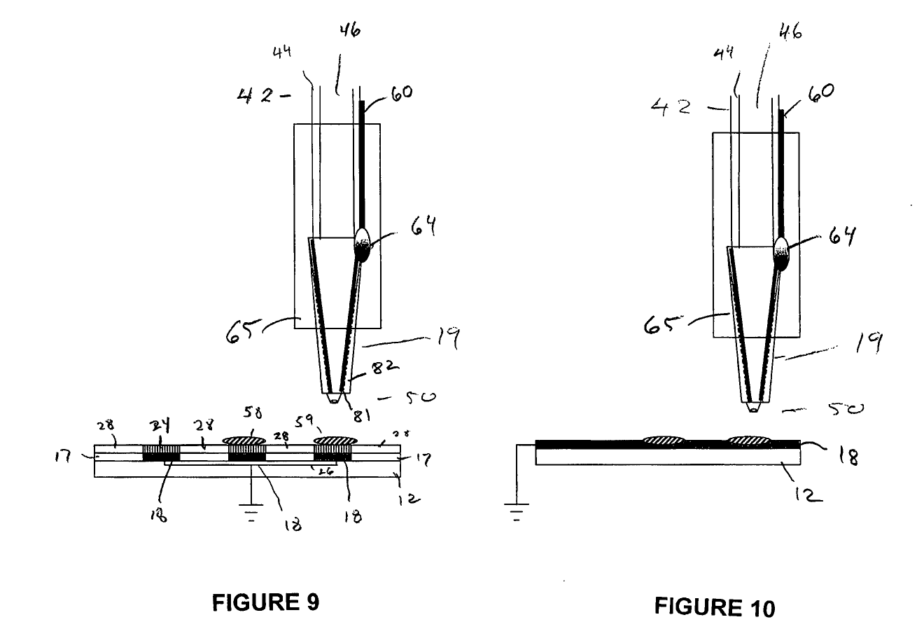

FIG. 9 illustrates another embodiment of a capillary electrophoresis tube 42 configured with the second electrode 19 at the proximal end 50 thereof. In this embodiment, the proximal end 50 of the capillary electrophoresis tube 42 is tapered so that at the proximal end 50 the capillary wall 44 has an outer diameter of about 40 microns or less, and the internal lumen 46 has a diameter of about 15 microns or less.

FIG. 9 further illustrates that any of the embodiments of the capillary electrophoresis tubes 42 configured with the second electrode 19 on the outer surface of the capillary wall 44 at the proximal end 50 thereof, the second electrode 19 can be made of a conductive layer of one or more metals deposited around the periphery of the proximal end 50 of the capillary electrophoresis tube 42 by a metal deposition processes. The conductive metal of the second electrode 19 may be attached to the lead wire 60 by bonding, soldering or adhesion though an electrically conductive adhesive 64. In an exemplary embodiment, the second electrode 19 on the proximal end 50 of the capillary electrophoresis tube 42 is comprised of a first metal layer 81, such as titanium, deposited by vapor deposition, sputtering, or other suitable method to form a bonding layer having a first thickness, for example, about 40 angstroms. The first metal layer 81 is overlaid with a second conductive metal 82 such as gold to a second thickness, for example about 80 angstroms or more, by a similar process. The deposition of the metal layers 81 and 82 forming the second electrode 19 is limited to the proximal end 50 by masking the outer wall 44 of the capillary electrophoresis tube 42 above the proximal end 50 during the deposition processes. Alternatively, or in addition, the capillary electrophoresis tube 42 may be oriented at an angle of 45 degrees or greater with respect to the direction of the deposition source so that only the proximal end 50 is contacted by the metal being deposited. In some embodiments, the metal(s) are deposited only on a portion of the proximal end 50 as illustrated, for example in FIG. 2 or 7. In other embodiments, the metal layer(s) are deposited evenly around the proximal end 50 in annular configuration, as illustrated for example, in FIGS. 9, 10, 15 and 21. The deposition of the metal layers 81 and/or 82 on the proximal end 50 of the capillary electrophoresis tube 42 can be evenly distributed around the proximal end 50 by rotating the capillary electrophoresis tube 42 during the deposition process. The capillary electrophoresis tube 42 is optionally surrounded by an insulating layer 65 near the proximal end 50.

Capillary electrophoresis tubes 42 having the second electrode 19 deposited on the proximal end 50 on the outer surface of capillary wall 44 have advantages in ease of construction, the ability to uniformly focus the electrical field across the proximal end 50 end, and in subsequent operation of the capillary electrophoresis system for separating collected components of the lysed cell. Because an electrical wire 62 is not within the lumen 46 of the capillary electrophoresis tube 42 as in the embodiment shown in FIG. 8, there is no electrical disturbance in the electrophoresis of material through the capillary electrophoresis tube 42. In addition, it is generally easier to attach the lead wire 60 to the outside surface of the capillary wall 44 than to fabricate a capillary electrophoresis tube 42 with the wire within the inner lumen 46 surface of the capillary. Finally, particularly with embodiments having a tapered proximal end 50, the second electrode 19 surrounding the periphery thereof promotes a more equal and focused distribution of electrical field though the selected cell.

FIG. 10 illustrates use of a capillary electrophoresis tube 42 having the second electrode 19 configured for fast electrical lysis in another embodiment of a system of the invention. In this embodiment of the system, the first electrode 18 is a transparent electrode made of a naked layer of metal or metals deposited on the top surface 16 the substrate material 12. The naked first electrode 18 is used directly as the surface that will contact the biological cells 58, 59. This embodiment lacks the non conductive layer 17, lacks an array of electrodes 30 and lacks the cell adhesion layer 22 or the hydrophilic regions 24 separated by hydrophobic regions 28. The cell is selected merely by positioning the capillary electrophoresis tube 42 with the second electrode 19 at the proximal end 50, and preferably with a tapered proximal end 50, within about 200 microns, or within 100 microns, or within 60 microns, or within 40 microns, or within about 20 microns of the selected cell 89. The selected cell 59 is lysed by applying a subsecond pulse of electrical field of about 10 to 15 Kv/cm (30-40 volts for a 20 micron gap) between the first 18 and second 19 electrodes located. Typically, the non selected cell 58 will not lyse with a subsecond pulse of the electrical field if the distance between the second electrode 19 and the non selected cell 58 is greater than about 200 microns or greater than about 300 microns or greater than about 500 microns.

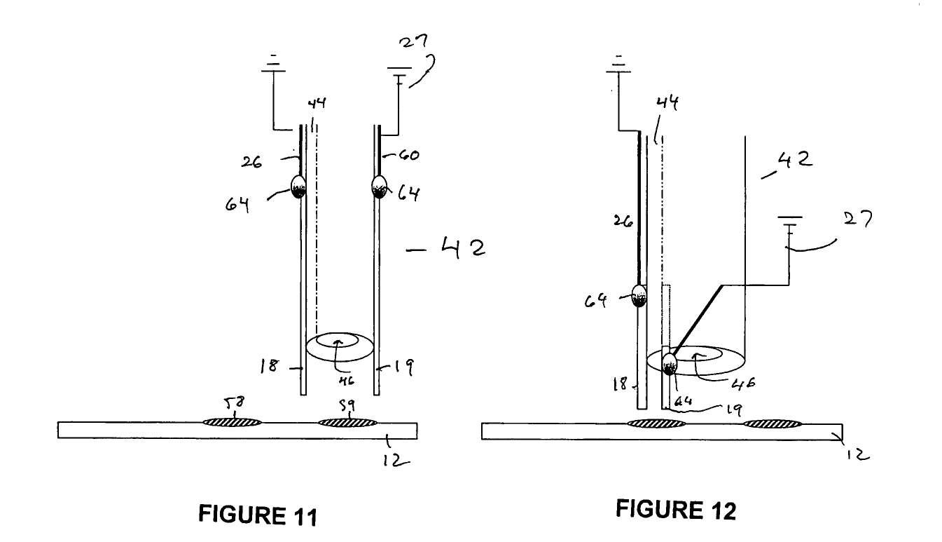

FIGS. 11 and 12 illustrate yet another aspect of the present invention, which includes a capillary electrophoresis tube 42 having a proximal end 50 configured with both the first 18 and second electrodes 19 for electrical lysis of the biological cell and collection of at least a portion of the contents thereof. In these embodiments, the lysing electrodes 18 and 19 are each positioned on the proximal end 50 of capillary electrophoresis tube 42 itself preferably within about 0 to 200 microns above or below the ultimate proximal end 50 of the capillary electrophoresis tube 42. This configuration differs in several respects from other systems for electrical lysis within a capillary as described by Xue and Yeung [10] or within a microfluidic device as described, for example, by Ramsey in U.S. Pat. Pub. No. 20030075446. With the present invention, there is no need for the cell to be in direct contact with the walls or interior of the micro-collection device 15 (i.e., capillary electrophoresis tube 42) prior to lysis. Lysis is conducted entirely outside the micro-collection device. In addition, there is no need for manipulations to draw the selected cell 59 into the capillary. Instead, the cell is ready for lysis merely by positioning the first electrode 18 and second electrode 19 within less than about 200 microns in one embodiment, or less than 100 microns, or less than 60 microns, or less than 40 microns, or about 20 microns or less of the selected cell Therefore, electrical lysis can be executed without perturbing the resting state of the cell by being loaded into a capillary or other microfluidic device. Moreover, there is no need to configure both of the lysing electrodes to be in conductive contact with the lumen of the microfluidic device. The capillary electrophoresis tubes 42 with both first 18 and second 19 electrodes on the bottom end are particularly usefully for selectively lysing non-adherent cells.

In FIG. 11, the first 18 and second 19 electrodes are in electrical contact with different poles of the electrical power supply or to ground. The electrodes 18 and 19 are mounted on the opposite sides of the exterior surface of the capillary wall 44 and separated from one another by a distance of less than about 200 microns in one embodiment, or less than 100 microns, or less than 60 microns, or less than 40 microns, or about 20 microns or less in various other embodiments. Preferably, but not necessarily, the lysing electrodes 18 and 19 extend beyond the proximal end 50 so that they can be positioned on either side of the selected cell 59. This embodiment is most suitable for capillary electrophoresis tubes 42 having a tapered proximal 50 end where the outer diameter of the capillary wall 44 is less than the foregoing separation distance between the first 18 and second electrodes 19.

In the embodiment illustrated in FIG. 12, the first lysing electrodes 18 is located on the exterior surface of the capillary wall 44 and the second lysing electrode 19 is located on the interior surface. This embodiment is more suitable for capillary electrophoresis tubes 42 lacking tapered ends, because the capillary wall 44 of most commercially available capillary electrophoresis tubes 26 typically has a thickness less than about 200 microns.

The close proximity of the first 18 and second 19 lysing electrodes in either of the embodiments depicted in FIGS. 11 and 12 provides the advantage of allowing lysis of a single selected cell on an ordinary cell chamber because the electric field is concentrated at the tip of the capillary electrophoresis tube 42 which is proximal to the selected cell 59 and distal from a non-selected cell 58. The cell chamber 10 itself need not be configured with electrodes.

The foregoing embodiments of the invention have focused on the use of single capillary electrophoresis tube for collection of cellular contents. In other embodiments, such as depicted in FIG. 21, and array 31 of capillary electrophoresis tubes 42 may be configured to correspond to the array of electrodes 30 and/or hydrophilic regions 24 on a cell chamber 10. In this embodiment, multiple cells 58, 59 may be selected for simultaneous lysis and collection in a high throughput screening system.

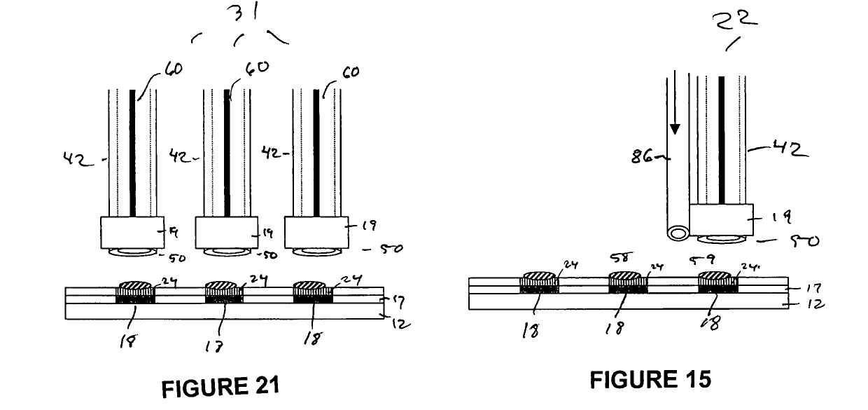

In another modification of the various apparatus of the invention, a single capillary electrophoresis tube 42 or array of capillary electrophoresis tubes 42 can be configured with adjacent micro-dispensing devices 86 such as illustrated in FIG. 15. The micro dispensing devices 86 may be a siphoning capillary, a reverse polarity capillary electrophoresis tube preloaded with a test substance, or a valve controlled capillary or nozzle for dispensing nanoliter volumes of test material into the medium 56 in the vicinity of the cell immediately adjacent to the capillary electrophoresis tube 42. In use of such a system 22, very small amounts of test material are introduced into the medium immediately prior to lysis of the selected cell 59 and collection of the contents thereof. Using such configurations, multiple cells on a single slide can be tested with different test materials in a high throughput assay system.

While a capillary electrophoresis tube 42 is a preferred type of micro-collection device 15 in various embodiments of the invention, as mentioned herein before, other micro-collection devices are also suitable for use and or configuration according to the practice of the invention. Examples of other suitable micro-collection devices include surface bound macromolecules 71 on an analytic “chips” such as described, for example, in U.S. Pat. No. 6,440,667. Still another type of suitable device includes optical fiber bundles bound to arrays of macromolecules 71 through a bead, as described for example in U.S. Pat. No. 6,429,027. Yet another type of device is a simple a carbon fiber, that is configured to detect the release of electrons from materials released from the lysed as described for example by Travis et al or Suaud-Chagnv et al [49, 50]. As illustrated in FIGS. 13A and 14A, a micro-collection device 15 such as chip 70 depicted in FIG. 13A, or fiber optic bundle 76 depicted in FIG. 14A, can be used without modification in combination with the cell chambers having an array of electrode pairs, such as illustrated for example, in FIG. 6. FIGS. 13B and 14B illustrate how these other micro-collection devices can be configured with single electrodes on their proximal ends analogous to the capillary electrophoresis tubes 42 depicted in FIGS. 2 and 7-10. FIGS. 13C and 14C illustrate that these micro-collection devices may also be configured to contain electrode pairs analogous to the capillary electrophoresis tubes 42 shown in FIGS. 11 and 12.

Another aspect of the invention is methods for lysing a biological cell using a single subsecond pulse of an AC or DC field across the biological cell using the various apparatus of the invention, and collecting at least a portion of the contents of the lysed cell thereof in a micro-collection device. In certain embodiments, the act of collecting is commenced simultaneously with the act of electrical lysis. In other embodiments, the act of collecting commences within 1 second, within 100 ms within 10 ms, within 1 ms, within 100 μs within 10 μs or within 1 μs of commencing the electrical lysis. Coordination between the timing of lysis and timing of collection can be accomplished using a timing control switch that activates the process of collection simultaneously, or within the foregoing prescribed times of commencing the electrical lysis. In preferred practices, the act of collecting uses a capillary electrophoresis tube with a proximal end positioned within the foregoing distance of the selected cell. The methods further include the act of separating at least a portion of the contents of the lysed cell in the capillary electrophoresis tube or other micro-collection device. Methods for controlling the timing between lysis, collection and separation of cellular contents using a capillary electrophoresis system, as well as configurations therewith using a microscope are described in the Examples that follow and in U.S. Pat. Nos. 6,156,576 and 6,335,201 where lysis is described using laser based lysis.

In preferred embodiments of methods of the invention, the selected cell 59 is positioned between the first electrode 18 and the second electrode 19. In other embodiments the selected cell 59 need not be positioned directly between the first 18 and second electrodes 19, but is positioned in close proximity thereto, within less than less than about 200 microns or less than 100 microns, or less than 60 microns, or less than 40 microns, or about 20 microns or from the center of the gap between the first 18 and second electrodes 19. The inter-electrode distance is likewise less than about 200 microns in one embodiment, or less than 100 microns, or less than 60 microns, or less than 40 microns, or about 20 microns or less in various other embodiments. Electrical lysis is commenced by applying an electrical potential of about 5 to about 30 Kv/cm in one embodiment, about 10 to 20 Kv/cm in another embodiment, or about 15 Kv/cm in another embodiment, between the first electrode 18 and the second electrode 19. The electrical potential is applied in a single pulse of an AC or DC field for a duration of about 10 milliseconds or less in certain embodiments, or 1 millisecond or less, or 100 microseconds or less in other embodiments or 10 microseconds or less in other embodiments.

In certain practices of the methods, the use of an AC field may provide advantages over DC fields, in that unlike DC fields, gas generation at electrodes employing an AC field (is not a significant issue. This property is important because bubbles formed at electrodes can be drawn into separation channels or into the capillary electrophoresis tube where they will interfere with electrophoresis. In addition, the use of AC fields prevents unwanted electrophoretic motion within the electrolyte during the time of lysis.

The devices, systems and methods of the present invention are suitable for use with both adherent cells and non-adherent cells. Adherent cells will tend to adhere to the surface of the cell container, especially over the hydrophilic regions 24. Non-adherent cells can be urged into a desired position between the first electrode 18 and the second electrode 19 using mechanical or non-mechanical tools. One example of a non-mechanical tool for this purpose is a laser microtweezer.

### EXAMPLES

### Example I

Materials and Methods for Electrical Lysis and Micro Collection of the Contents of a Cell In a Capillary Electrophoresis Tube

Reagents. Oregon Green 488 carboxylic acid diacetate (6-isomer) (Oregon Green diacetate), 5-carboxyfluorescein diacetate and 6-carboxyfluorescein diacetate (carboxyfluorescein diacetates) were purchased from Molecular Probes (Eugene, Oreg.). Oregon Green free acid and carboxyfluorescein free acids were obtained by hydrolyzing the diacetate forms of the fluorophores in Na2CO3 solution (pH 10) at 37° C. overnight. Tissue culture materials were obtained from Gibco BRL (Gaithersburg, Md.). Acrylamide, ammonium persulfate and TFMED were bought from Bio-Rad Laboratories (Hercules, Calif.). Methacryloxy-propyltrimethoxysilane (MAPS), was obtained from United Chemical Technologies, Inc. All other chemicals were from Fisher Scientific (Pittsburgh., Pa.). The buffer, ECB, was composed of 135 mM NaCl, 5 mM ICI, 10 mM HEPES, 2 mM MgCl2 and 2 mM CaCl2 adjusted to pH 7.4 with NaOH.

Coating Capillaries with Polyacrylamide. Fused-silica capillaries (50-μ i.d., 360-μ o.d.; Polymicro Technologies, Phoenix, Ariz.) were coated with polyacrylamide. Briefly the capillary was washed with 1 M HCl (1 hr), H2O (10 min), 1 M NaOH (1 hr), and H2O (10 min). The capillary was then washed with methanol (10 min), and filled with 50% MAPS (in methanol). Both ends of the capillary were sealed and the capillary was placed at room temperature for 12-15 hrs. After cleaving the sealed ends of the capillary, it was rinsed with methanol followed by water, each for 10 min. The capillary was filled with freshly prepared reaction buffer (3% acrylamidc, 0.6% ammonium persulfate and 0.2% TEMED in water). After 3 hrs, the capillary was rinsed with water for 10 min and dried with nitrogen for 10 minutes. Prior to use the capillary was rinsed with ECB.

Cell Culture. Rat basophilic leukemia (RBL) cells were used as the model cell for this study. These cells were grown at 37° C. in a humidified 5% CO2 atmosphere in Dulbecco's modified eagle medium (DMEM) supplemented with fetal bovine serum (10%), and L-glutamine (584 mg/L). Penicillin (100 units/mL) and streptomycin (100 μg/mL) were added to the media to inhibit bacterial growth. The cells were grown in a cell chamber constructed by using Sylgard (Dow Corning, Midland, Mich.) to attach a silicon “O” ring (15/16-in. outer diameter) to a 25-mm, round, no. 1, glass cover slip. The cover slip was coated with titanium and gold as described below. The cells were allowed to grow for 12-24 h prior to use. Cells were plated at concentrations determined empirically to produce approximately one cell per 1000× field of view on the day of the experiment. For experiments the growth media was removed front the cell chamber and replaced with ECB.

Preparation of Electrically Conductive Coverslips. To make the cover slips (i.e, substrate 12) conductive, they were coated first with 50 A of titanium followed by 80 A of gold with an E-beam vacuum evaporator. The purpose of the initial titanium layer was to provide a surface to which the gold could strongly adhere. To prepare the cover slips for coating the following procedure was used: i) clean in an ultrasonic bath containing detergent solution (Alconox, 0.5% (weight/volume)) for 15 minutes, ii) rinse with water, iii) soak in 30% HCl for 15 min, iv) rinse with water, v) dry with filtered, compressed nitrogen, and vi) bake for 30 min at 120° C. Metal-coated cover slips had a resistance of about 15-20 ohms/cm. The transparency of the cover slips was compromised slightly by the metal layers; nevertheless, imaging of the cells through the coated cover slip with an inverted microscope was easily performed. A short length of platinum wire was attached to the gold layer of the cover slip with a conductive epoxy and used to connect the metal coating on the cover slip to the pulse generator.

Loading Cells with Fluorescent Compounds. 6-carboxyfluorescein diacetate, 5-carboxyfluorescein, and/or Oregon Green diacetate were loaded into the cells for these experiments. These dyes are membrane permeant but are not fluorescent until they are hydrolyzed into free acids by ubiquitous intracellular esterases. The free acids are trapped within the cells, although cells do transport the dye out over time via anionic transporters and other as yet undefined mechanisms. Carboxyfluorescein diacetates and/or Oregon Green diacetate were dissolved in ECB with glucose (10 mM) and immediately added to the cell chamber. When both fluorophores were loaded into the same cell, the concentration of the two carboxyfluorescein diacetates and Oregon Green diacetate was 4, 4, and 1 μM, respectively. When Oregon Green diacetate was the only fluorophore loaded, the concentration was 0.5 μM. Cell chambers containing the diacetates were incubated in the dark for 30 min at room temperature. The cells were then washed five times in ECB and were used within −5-10 min. After loading cells with fluorophores, a constant flow of FCB (5.5 ml/min) through the cell chamber was utilized to remove fluorescent dye transported from the cells into the surrounding buffer. Since the cell chamber served as the inlet reservoir during electrophoresis, continual replacement of the chamber buffer was required to avoid contamination of the capillary with fluorophore extravasated from the intact cells remaining in the cell chamber during experiments.

Cell Imaging. An inverted fluorescence microscope with a 100× oil-immersion objective (Diaphot 200, Nikon, Melville, N.Y.) was used for transmitted light and fluorescence imaging (ex/cm 488/514 nm). Images were digitized by a Hitachi CCD camera (Model KP-MIAN) and recorded with a video cassette recorder. Images were collected at standard video rate (30 frames/s), thus the time interval for each frame was 33 ms.

Production and Metal-Coating of Tapered Capillary Tips With an Electrode at the Proximal End One end of the capillary was pulled by a capillary puller (Model P-2000, Sutter Instrument Co. Novato, Calif.) to form a sharp tip. The tip was carefully cut with a ceramic stone under a dissecting microscope to form a tip of ˜30-40μ in outer diameter. The internal diameter was 10-15 μm. The capillary was then attached to a syringe containing pressurized water so that water flowed through the capillary during subsequent steps. The capillary tip was washed with water and placed in an ultrasonic bath containing a detergent solution (Alconox, 0.5% (weight/volume)) for 5 min. After which, the capillary tip was washed with methanol and then water. The syringe of pressurized water was then detached born the capillary and the capillary was dried under a stream of nitrogen. During metal coating, the body of the capillary was coiled and covered with polyimide tape to avoid metal deposition in unwanted regions. The outer wall of the capillary tip was coated sequentially with titanium and gold by E-beam evaporation for a 5-6 cm length to form the electrode. The capillary tip was oriented at a 45° angle with respect to the metal source so that the end and one side of the capillary were coated while the opposite side of the capillary was not coated with metal. The capillary end and exposed side were coated with 50 Å of titanium followed by 80 Å of gold. A platinum wire was glued to the metal-coated portion of the capillary with conductive epoxy, and was surrounded by a small plastic pipette tip. A large plastic pipette tip was also employed to insulate the small pipette tip. The two ends of the large pipette tip were, sealed by a nonconductive epoxy to fix the electrode/capillary in place.

Cell Lysis By An Electric Field. The gold-coated capillary tip was mounted perpendicularly to the cover slip on a 3-axis micromanipulator (World Precision Instruments, Sarasota, Fla.). The micromanipulator enabled precise positioning of the capillary lumen over the cell selected for analysis. A pulse generator (model HP 214B, Hewlett-Packard, Palo Alto, Calif.) was used to apply a voltage pulse between the capillary and coverslip. The pulse generator provided pulses in the shape of a square wave with variable strength and width and was operated in burst mode at 1 pulse per burst. The pulse was triggered by an external voltage (5 volts) generated by an A/D board (KPCI 3100, Keithley Instruments, Cleveland, Ohio) installed in a computer (Dell Computer, Round Rock, Tex.).

Capillary Electrophoresis. A high-voltage power supply (CZE1000R, Spellman, Plainview, N.Y.) was used to apply a voltage across the capillary for electrophoresis. The capillary (50μ inner diameter, 380μ outer diameter) possessed a total length of 75 cm. The electrophoretic buffer was ECB and was chosen to mimic the ionic composition of a physiologic extracellular fluid. The cell chamber served as the inlet reservoir and was held at ground potential. The outlet reservoir was held at a constant voltage between +12 and +14 kV as indicated. A custom computer program written in TestPoint (Keithley Instrument, Inc., Cleveland, Ohio) was utilized to simultaneously trigger the voltage pulse for cell lysis and initiate the electrophoresis. The inlet and outlet reservoirs were at equal heights to avoid gravity flow. Free acid standards of carboxyfluorescein and Oregon Green were loaded into the capillary by gravity-induced hydrodynamic injection.

Fluorescence Detection. A 1 cm optical window in the capillary was created by burning off the polyimide coating 50 cm from the inlet. The capillary lumon was interrogated by the focused laser beam of an argon ion laser (488 nm, Uniphase, San Jose, Calif.). The focusing long possessed a focal length of 5.0 cm (CVI, Albuquerque, N. Mex.). Fluorescence was collected at a right angle to the capillary and laser beam with a microscope objective (40×, 0.75 n.a., Plan Fluor, Nikon, Melville, N.Y.). The light was collected and measured with a photomultiplier tube (PMT) (8928, Hammamatsu, Bridgewater, N.J.) after filtering with a 488 notch plus filter (Kaiser Optical Systems, Ann Arbor, Mich.), and a band-pass filter (535DF50, Chroma, Brattleboro, Vt.). A spatial filter was placed in front of the filters to remove the scattered laser light. The PMT current was amplified and converted to a voltage with a preamplifier (Model 1212, DL Instruments, Dryden N.Y.). The signal was digitized by a data acquisition board (KPCI 3100, Keithley Instruments, Inc., Cleveland, Ohio). The data were plotted using Origin (Microcal, Northhampton, Mass.).

Quantitation of the Amount of Cellular Fluorophore Loaded into a Capillary. Cells were loaded with Oregon Green as described above. Two sets of experiments were performed simultaneously. In one set of experiments, single cells were lysed and loaded into the capillary of an LMS. The area of the peak obtained from each cell was then compared to that of standards to determine the number of moles of Oregon Green collected from the cell. With the LMS, the standards were loaded by hydrodynamic flow for 7 seconds with a height difference between the inlet and outlet reservoir fluid levels of 2.5 cm. The volume loaded was calculated using Poiseulle's equation. The number of moles was then obtained from the known concentration of the sample and the volume loaded.

In the second set of experiments, single cells were lysed and loaded into a capillary using the electrical lysis system (1 ms pulse duration, 40 V, 20 μm inter-electrode distance). The area of the peak obtained from each cell was then compared to that of standards to determine the number of moles of Oregon Green collected from the cell. The moles of standard loaded into the capillary was calculated from the concentration and the volume loaded. In contrast to the LMS, a simple Poiseulle equation could not be used to calculate the volume of standard loaded into the capillary since the capillary lumen was tapered at one end.

In order to determine the volume of standard loaded under a specific set of hydrodynamic conditions, a concentrated Oregon Green sample of known concentration was hydrodynamically loaded into the capillary (used for electrical lysis). The hydrodynamic force was created by placing the capillary inlet into the sample solution and elevating the level of the inlet solution 7 cm above the level of the outlet solution for 5 s. In one instance the sample was electrophoresed and the peak area measured. In a second trial, the concentrated Oregon Green sample was loaded in an identical fashion but the sample was not electrophoresed. Instead, the loaded sample was pushed back, out of the capillary into a collection tube and diluted to a known final volume (50 μl). This diluted sample was then loaded into the capillary under conditions identical to the concentrated sample and electrophoresed. All electrophoretic and detector conditions were identical to that when the concentrated sample! of Oregon Green was electrophoresed.

Because the peak area is proportional to the number of electrophoresed moles and the concentrated and diluted samples possessed identical injection volumes, the ratio of the peak areas of the two samples yields the degree of dilution of the diluted Oregon Green (i.e. C_{c}Vᵢₙⱼ/A_{c}=C_{d}Vᵢₙⱼ/A_{d} for Vᵢₙⱼ, the injection volume; C_{c}, and C_{d}, the concentration of the concentrated and dilute Oregon Green samples, respectively; A_{c} and A_{d}, the peak area of the concentrated and dilute Oregon Green samples, respectively). The degree of dilution (C_{d}/C_{c}=A_{d}/A_{c}) can then be used to calculate the injection volume since the final volume of the diluted sample was known (i.e. A_{d}/A_{c}=Vᵢₙⱼ/V_{final} for V_{final}, the final volume of the diluted sample).

### Example II

Results of Subsecond Electrical Lysis and Collection of Cellular Contents in a Capillary Electrophoresis Tube

The goal of these experiments was to determine whether fast electrical lysis of a cell in combination with electrophoresis of the cell's contents in a capillary might be compatible with the study of fast signaling reactions in cells. To be successfully used to follow rapidly changing cellular reactions, the method should preferably have the ability to terminate the cellular chemical reactions of interest on time scales less than one second, or less than 500 ms, or less than 100 ms and preferably, less than ˜33 ms. The actual time of lysis may be lower than 33 ms, which is the lower limit of measurability in this example because that is the period that elapses between frames of a video recorder. For many cellular reactions such rapid lysis can be achieved by lysis of the cell on similar time scales. [11,12]. Turbulence from the lytic process and diffusion of the reactants can then greatly slow the rate of many chemical reactions essentially terminating the reaction. A second desired requirement is that the method be capable of selecting a single cell for analysis. Since individual cells are asynchronous in their response to stimuli, loading the contents of multiple cells at once into the capillary would yield a population average which is frequently not representative of the behavior of a single cell. Since most mammalian cells grow attached to a surface and removal from that surface disrupts or alters multiple signaling pathways, the lytic method must not require removal of the cell from the surface prior to lysis nor can it disturb or stress the cell prior to the moment of lysis (since this can also activate signaling pathways). [30-34]. Lastly, the strategy for cell lysis must be compatible with capillary electrophoresis. Efficient loading of the cellular contents into a capillary electrophoresis tube without deterioration of the subsequent electrophoretic separation is most desirable.

Design/Fabrication of the Capillary Electrode. Lysis of cells in an electric field is due to dielectric breakdown of the membrane as the transmembrane voltage reaches a certain magnitude. In mammalian cells membrane breakdown leading to pore formation begins at a transmembrane voltage of 0.2-1.5 V depending on the conditions and the cell type. [13-18]. Limited numbers of small pores are reversible and this is the basis for electroporation. As the transmembrane voltage is increased, larger pores and greater numbers of pores form creating irreversible holes in the membrane lysing the cell.

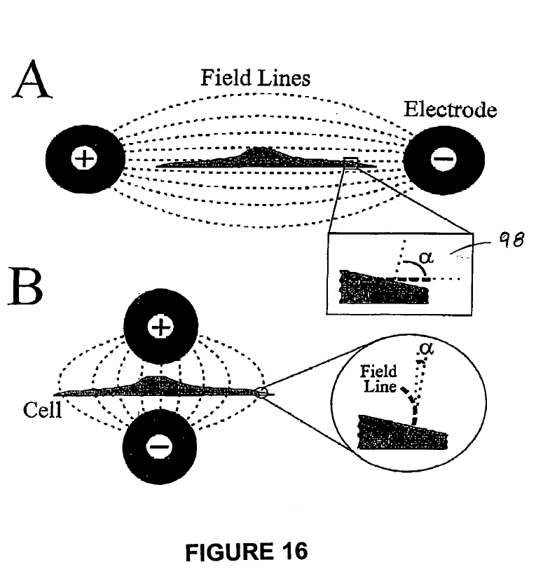

In designing the electrodes the goal was to utilize an electrode geometry that leads to the fastest formation of irreversible pores at the lowest inter-electrode voltage. Two different electrode placements with respect to the adherent cell are possible as illustrated in FIG. 16. The potential across the plasma membrane of the cell depends on the cosine of the angle (α) 98 between the electric field lines 97 and a line 100 drawn perpendicular to the surface of the membrane 102 of the cell 59. [13-18]. Thus, the potential drop across the membrane is maximal for field lines perpendicular to the cell surface and minimal for field lines nearly parallel to the cell surface. When electrodes are placed to the right and left of a cell as illustrated in FIG. 16A, the field lines 97 or direction of the electric field is parallel to the surface of the flattened, adherent cell 59. For this geometry, the electric field is less efficient at creating pores throughout the membrane. In contrast, electrodes positioned above and-below the cell are more efficient in creating a high transmembrane voltage throughout large areas of the plasma membrane as depicted in FIG. 16B.

Multiple investigators have demonstrated that pores initially form in the cell membranes facing the electrodes rather than,—in the membrane regions away from the electrodes. [35-38]. For example in flattened, adherent cells with electrodes placed to the right and left of the cell as in FIG. 16A pores initially formed in the outermost tips of the cell facing the electrode.[35]. Additionally a number of investigators have also demonstrated that pores form at a lower inter-electrode voltage in the membrane facing the positive electrode (the hyperpolarized portion of the cell's membrane) than in the membrane facing the negative electrode (depolarized membrane). The reason for this phenomena is not known but suggests that the positive electrode should be positioned above the cell. Thus the largest and earliest pores will form in the cellular membrane facing the buffer solution rather than in the membrane regions tightly adhered to the surface of the cell chamber. This would yield the fastest release of the cell's contents into the buffer solution contained just below the lumen of the capillary. For these reasons the design shown in FIG. 16B was selected for testing as the geometry most likely to give fast cell lysis at the lowest possible inter-electrode voltage. However, designs with the electrodes placed adjacent to the cells as depicted in FIG. 16A are may also be effective, particularly when the cells have a spherical geometry, as occurs, for example in many blood cell types.

The design of FIG. 16B requires that electrodes be placed above and below the cell and that each electrode be in close proximity to the cell. Further the electrodes must be capable of lysing only one cell yet any cell in the chamber must be selectable for lysis and analysis. To meet those requirements one electrode was fabricated on the substrate on which the cell grew and the second electrode was placed on the tip of the capillary as depicted in FIG. 10. A thin layer of titanium and then gold was evaporated with a glass coverslip. The coverslip was assembled into a cell chamber and the cells cultured on the metal coated coverslip. The cells attached and grew as well on the metal-coated coverslip as! on the glass surface. Since the cover slip-based electrode extended throughout the cell chamber, the second or upper electrode needed to confer both the ability to lyse a single cell and the ability to select any cell in the chamber. To meet this need, one end of a capillary was pulled to a fine tip (30-40μ outer diameter) using a laser-based capillary puller. The tip was then coated with a thin layer of titanium and gold. A platinum wire was used to connect the metal-coated region of the capillary to a pulse generator. Placing the small capillary tip in close proximity to the cell and underlying electrode should largely confine the electric field to the region between the capillary tip and the immediately adjacent surface of the coverslip. The capillary was also mounted on a micromanipulator so that it could be positioned over any cell contained in the chamber.

Influence of the Magnitude of the Voltage Pulse on Cell Lysis. For this application, termination of the cellular reactions depends on the rate of lysis of the cells. For this reason, we initially determined how fast a cell could be lysed by a voltage pulse. In addition since electroporation depends on both the magnitude and the duration of the voltage pulse, we also sought to determine; how these parameters influenced the rate of cell lysis. Cells were loaded with the fluorophore Oregon Green. Oregon Green is trapped in the cytoplasm of cells as long as the plasma membrane remains intact. The fluorescence of Oregon Green in a cell was measured before during and alter application of a voltage pulse to a cell. The time for complete loss of Oregon Green fluorescence from the cell (as judged by fluorescence microscopy) was used as a measure of how fast lysis occurred, i.e., the time for leakage of the cell's contents.

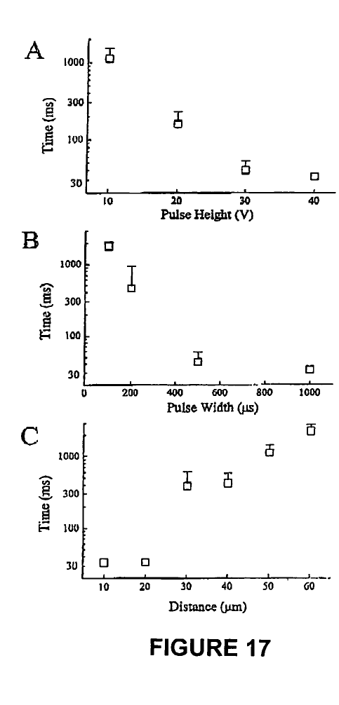

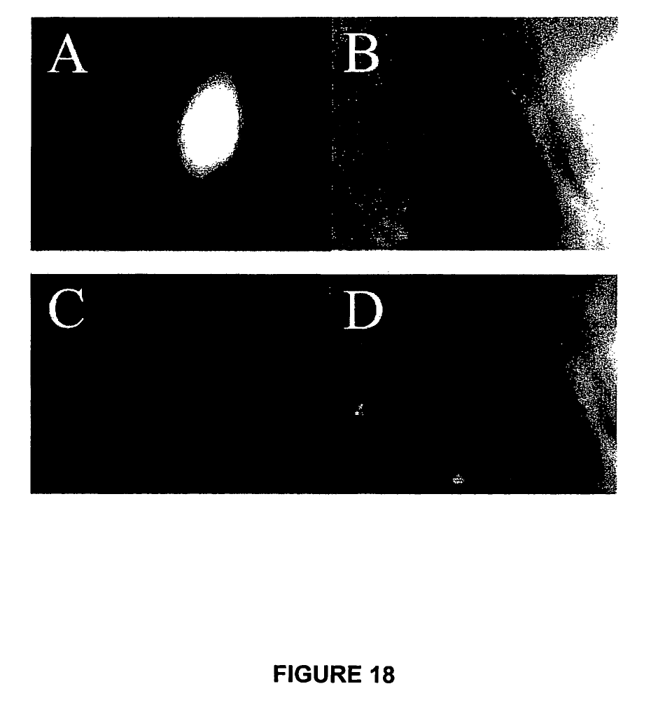

The influence of the magnitude of the voltage pulse was investigated by varying the voltage while keeping all other parameters fixed. Four different pulse strengths between 10 and 40 V were tested with a pulse duration of 1000 μs and an inter-electrode distance of 20 μm. Six cells were lysed at each voltage, and the tune for the fluorescence to decrease to background was plotted as a function of the voltage as shown in FIG. 17. Error bars were plotted to show the standard deviation. At the lowest voltages (10-20 V), the efflux of the Oregon Green from the cells was slow requiring hundreds of milliseconds to return to the background level of fluorescence. From the transmitted-light images, the profiles of the cells could still be observed. These cells possessed the morphology of a dead cell (refractile, granular appearance) suggesting that only remnants of the cytoskeleton, plasma membrane, and some organelles remained. These findings suggest that the cells were slowly lysed following the voltage pulse. This may have been due to the formation of membrane pores of limited size and number requiring long times for complete efflux of intracellular molecules. The large error bars are due to the cell-to-cell variability in the voltage intensity required to lyse or permeabilize cells. [23,27,35]. This variability may be due to differences in the cellular morphology, cell cycle, and other factors. When the voltages were 30 V and greater, almost all cells were lysed within one image frame, i.e. 33 ms as illustrated in FIGS. 18A,B. The transmitted-light images also showed that very few remnants of the cells remained attached to the substrate as shown in FIGS. 18C,D. At these higher voltages, electrolysis of water at the electrodes was also increased as evidenced by larger numbers and sues of gas bubbles forming on the electrodes. These field strengths (15 kV/cm) are similar to that described previously to cause cellular lysis in the context of electroporation, and are approximately 10 fold greater than that needed to permeabilize cells. [13-18].

Influence of Pulse Duration on Cell Lysis. In a second set of experiments, the influence of the duration of the voltage pulse was investigated by fixing all parameters except the pulse length. Six different pulse durations (1 μs, 10 μs, 100 μs, 200 μs, 500 μs, and 1000 μs) were tested while the voltage was fixed at 40 V and the inter-electrode distance was again 20 microns. Six cells were lysed for each pulse length, and the time for the fluorescence to decrease to background was plotted as a function of the pulse width as shown in FIG. 17B. Error bars were plotted to show the standard derivation. Cells receiving the 1 μs and 10 μs long pulses showed a decrease in fluorescence. However, during the time that the cells were examined, the fluorescence did not approach the background fluorescence level suggesting that the cells were not lysed. Most likely the cells were reversibly electroporated during exposure to these brief voltage pulses. Since the capacitance of the system was unknown, it was possible that the peak voltage of these shorter pulses did not reach 40 V. For this reason a digital oscilliscope was used to determine the true pulse width and magnitude when the voltages were applied across the capillary and coverslip electrodes. For a selected pulse duration of 1 μs and voltage of 40 V, the true peak voltage was only 30 V with a width of 1 μs. For a selected width of 10 μs and voltage of 40 V, the actual voltage pulse was square in shape with a width of 10 μs and a magnitude of 40 V. The capacitance of the system therefore did not blunt the magnitude or duration of the applied 10 μs-voltage pulse.

Longer voltage pulse durations (100-1000 μs) resulted in the complete loss of cellular fluorescence over time after application of the pulse. For pulse widths of 100 and 200 μs the cellular fluorescence required hundreds of milliseconds to return to the background level as shown in FIG. 18. Cellular ghosts or remnants of the cytoskeleton, plasma membrane, and organelles remained visible after application of the voltage pulse. These results suggest that the cells were slowly lysed at these pulse duration. For longer pulse widths (≧500 μs), the cells were lysed more completely and rapidly. Nearly all cells had complete loss of fluorescence within 33 μs. Transmitted light images showed no or few cell remnants attached to the coverslip. Thus, cells could be rapidly lysed with single, brief voltage pulses.

Influence of Inter-Electrode Distance on Cell Lysis. The degree of electroporation of a cell depends on the Inter-electrode distance when the absolute voltage and pulse duration are maintained constant. For this reason, we sought to determine how the inter-electrode distance influenced the rate of cell lysis when both the voltage and the pulse duration were fixed. Cells were loaded with Oregon Green and the time for fluorophore efflux was measured as the interelectrode distance was varied. As seen in FIG. 17C the greater the inter-electrode distance, the slower the cell lysis. For an inter-electrode distance between 10-20 μm, the cells were nonfluorescent within 33 ms suggesting that they were very rapidly lysed. It took substantially longer (˜330 ms) for Oregon Green to exit the cell when the inter-electrode distance was 30-40 μm. At these distances the ghosts or profiles of the dead cells were visible by transmitted light microscopy. For these voltages and pulse widths, the required distance between the capillary and cell was much smaller than that required for laser-based lysis (˜60 μm) making the alignment of the capillary over the cell slightly more difficult for electrical lysis. [11]

Variability of the Capillary-Based Electrode. The properties of each fabricated capillary/electrode combination were slightly different. This can be seen in FIG. 17, panels A and C. In FIG. 17A, release of Oregon Green occurs within 33 ms when the average electric field between the electrodes is as low as 1.5 V/μm. However in FIG. 17C the release of the Oregon Green within 33 ms does not occur until the field reaches an average of 2 V/μm. This variability is most likely due to differences in the manufacturing process. After the capillary tip was pulled, it was manually cleaved to yield an outer diameter varying between 30 and 40 μm at the very tip. In addition to the variability in the exact tip diameter, the shape of the tip was also difficult to control with this process. Further optimization of the tip pulling protocol might eliminate the manual cleavage procedure. Nevertheless with the current manufacturing process, each capillary/electrode combination was easily tested for the minimum average electric field required to lyse all cells within 33 ms. Cell lysis in subsequent experiments was then performed with the electric field optimal for that particular metal-coated capillary tip.

On average the electrode fabricated on the capillary could be used ˜50 times before loss of the gold/titanium coating became significant. This was most likely due to two factors: i) electrolytic removal of gold from the capillary/electrode surface during cell lysis, and ii) electrolytic removal of titanium due to pin holes in the gold coating. Due to the relatively large voltages applied across the electrodes during cell lysis; oxidation of gold and exposed titanium is expected to occur at the anode on the capillary. [39]. With each brief voltage pulse, the anode will loose gold and/or titanium until eventually all of the metal is removed. After prolonged usage, large flakes of metal were observed detaching from the glass surface. This suggests that the underlying titanium might have undergone oxidation first releasing the gold from the surface. It may be possible to minimize metal “lilt-off” from the glass surface by depositing a thicker gold coating to cover pinholes. With the current method one side of the capillary is in a “shadow” during metal deposition and so is poorly coated or uncoated. The boundary region between this poorly coated side and the heavily-coated capillary regions (the end and opposite capillary side) may leave exposed titanium which can be electroplated from the glass undercutting the adjacent more heavily coated regions of the capillary. Rotating the capillary during metal deposition would eliminate the shadowed side of the capillary and potentially increase its lifetime.

Capture and Separation of a Cell's Contents. During electrical lysis of a cell, the high voltages applied across the electrodes can generate several gases. Oxidation of chloride and water at the anode can generate chlorine and oxygen, respectively, while reduction of water at the cathode can generate hydrogen gas. [39]. Consequently, a substantial amount of turbulence might occur near the electrodes as gas is generated and released. A small amount of turbulence is desirable since it promotes dilution of the cell's contents essentially terminating most cellular reactions. However, excessive mixing can lead to extreme dilution making the cellular contents undetectable or to an inability to collect the cell's contents into the capillary lumen. Further, gas generation at the electrodes might enter the capillary blocking or impairing analyte movement through the capillary during electrophoretic separation.

To determine whether a cell's contents could be collected and detected following electrical lysis, cells were loaded with Oregon Green. The capillary was then positioned 20 microns above the cell with the capillary lumen centered over the cell. The cell was lysed by a single electrical pulse (30-40 volts, 1 ms pulse width). A continuous high voltage was applied across the capillary ends simultaneously with the lytic voltage pulse. The fluorescence of analytes moving through the capillary was then monitored. A standard of Oregon Green was used to identify the migration time of Oregon Green in the capillary. One peak at a migration time nearly identical to the migration time of the Oregon Green standard was observed (n>20) as shown in FIGS. 18A,B Since no hydrodynamic flow was present, the contents of the cell were most likely loaded into the capillary by a combination of both electrophoretic movement and electroosmotic fluid flow. The average number of theoretical plates (±standard deviation) was similar for the standard and cell-derived peaks, 214,972±30,485 (n=8) and 197,109+29,113 (n=8), respectively. The similar number of theoretical plates suggests that the sample derived from the cell was not extensively diluted during cell lysis.

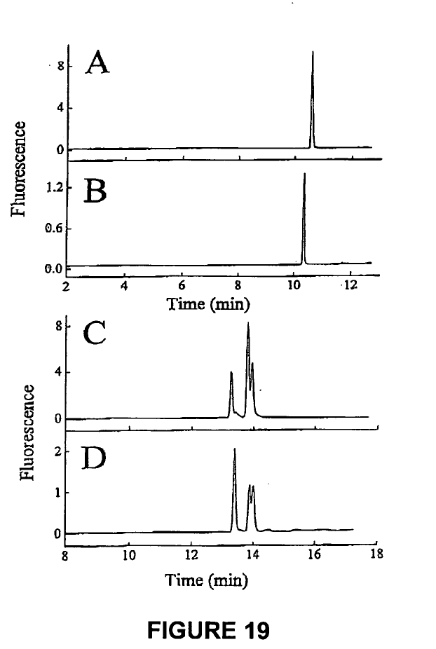

To determine whether the contents of an electrically lysed cell could be separated, cells were loaded with Oregon Green, 5-carboxyfluorescein, and 6-carboxyfluorescein. As described above, the cell was lysed and loaded into the capillary and the contents electrophoresed through the capillary. For all cells (n>5), the electropherogram displayed three peaks with migration times nearly identical to that of a standard composed of Oregon Green, 5-carboxyfluorescein, and 6-carboxyfluorescein as shown in FIGS. 19C,D. As a negative control, a RBL cell not loaded with any of these fluorophores was lysed and analyzed in the same manner. In this case, no peak was detected.

Collection Efficiency of the Cellular Contents. To estimate the loading efficiency of the cellular lysate into the capillary following electrical lysis, the number of moles of Oregon Green obtained from; a cell that was electrically lysed was compared to that from a cell lysed with the laser-induced shock wave. Cells treated in an identical fashion except for the method used to lyse the cells, should contain on average the same number of moles of fluorophore. Since the loading efficiency of the laser-based strategy has been measured, comparison of the numbers of moles collected from cells lysed by the two methods should yield an estimate of the collection efficiency following electrical lysis.

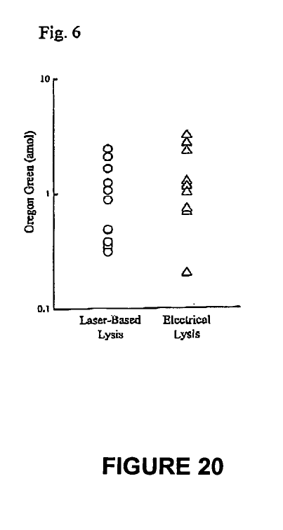

For the experiment, cells were loaded with fluorophore and then split into two portions. One group of cells was utilized in experiments on a laser micropipette system and single cell lysis was performed as described previously with a laser-generated shock wave. A second group of cells was electrically lysed. In both instances, the contents of the cells were loaded into an overlying capillary and electrophoretically separated. The area of the peak on the electrophoretic trace was then compared to that of standards to determine the number of moles of Oregon Green loaded from the cell. Eleven cells were electrically lysed while thirteen cells were analyzed with the LMS. Due to the cell-to-cell variability in the concentration of the fluorophore loaded into the cell, large standard derivations were obtained in both cases. For this reason, each single-cell measurement was plotted rather than the mean values, which is depicted in FIG. 20. The distribution in the number of moles of Oregon Green was similar for both lytic methods; therefore, the collection efficiency of the two methods was likely to be similar. Previously, Sims and colleagues demonstrated that the efficiency of collection for the LMS was close to 100%. [11] These results suggest that collection of the cellular contents into the capillary following electrical lysis was highly efficient.

Various embodiments of the invention have been described in particular figures and certain places in the text. The invention is not limited to these particular embodiments singularly, but also includes systems having any combination of the various embodiments that are not mutually exclusive of one another in functional operation. For example, the array of capillary electrophoresis tubes depicted in FIG. 21 may be configured without electrodes, or with any of the embodiments of electrodes described in the other Figures.

Insubstantial changes from the claimed subject matter as viewed by a person with ordinary skill in the art, now known or later devised, are expressly contemplated as being equivalently within the scope of the invention and its various embodiments. Therefore, obvious substitutions now or later known to one with ordinary skill in the art are defined to be within the scope of the defined elements. The invention and its various embodiments are thus to be understood to include what is specifically illustrated and described above, what is conceptually equivalent, what can be obviously substituted and also what essentially incorporates the essential idea of the invention.

Many alterations and modifications may be made by those having ordinary skill in the art without departing from the scope of the invention. Therefore, it must be understood that the illustrated embodiments have been set forth only for the purposes of example and that it should not be taken as limiting the invention except in accordance with the claims that follow and equivalents thereof.

### REFERENCES

(1) Yeung, E. S. J. Chrom. A 1999, 830, 243-262.

(2) Jankowski, J. A.; Tracht, S.; Sweedler, J. V. Trends Anal. Chem. 1995, 14, 170-176.

(3) Zabzdyr, J. L.; Lillard, S. J. Trends Anal. Chem. 2001, 20. 467-476.

(4) Stuart, J. N.; Sweedler, J. V. Anal. Bioanal. Chem. 2003, 375, 28-29.

(5) Chen, G.; Ewing, A. G. Critical Rev. Neurobiol. 1997, 11, 59-60.

(6) Cannon, D. M.; Winograd, N.; Ewing, A. G. Annu. Rev. Biophys. Struct. 2000, 29, 239-263.

(7) Luzzi, V.; Lee, C. L.; Allbritton, N. L. Anal. Chem. 1997, 69, 4761-4767.

(8) Cruz, L.; Moroz, L.; Gillette, R.; Sweedler, J. V. J. Neurochem. 1997, 69, 110-115.

(9) Olefirowicz, T. M;; Ewing, A. G. Anal. Chem. 1990, 62, 1872-1876.

(10) Xue, Q; Young. E. S. Journal of Chromotogr. B. 1996, 677, 233-240.

(11) Sims, C. E.; Meredith, G. D.; Kmsieva, T. B.; Berns, M. W.; Tromberg, B. J.

Allbritton, N. L. Anal. Chem. 1998, 70, 4570-4577.

(12) Li, H.; Sims, C. E.; Wu, H, Allbritton, N. L. 2001, Anal. Chem. 73, 4625-4631.

(13) Weaver, J. C. Journal of Cellular Biochemistry 1993, 51, 426-435.

(14) Rae, J. L.; Levis, R. A. Eur. J Physiol. 2002, 443, 664-670.

(15) Ho, S. Y.; Miltal,: G. S. Critical Rev. Biotech. 1996, 16, 349-362.

(16) Tsong, T. Y. Biophys. J 1991, 60, 297-306.

(17) Bilska, A. Q.; DeRruin K. A.; Krassowska, W. Bioelectrochemistry 2000, 51, 133-143. (18) Wilhelm, C.; Winterhalter, M.; Zimmermann, U.; Benz, R. Biophys. J. 1993, 64, 121-128.

(18) Wilhelm, C.; Winterhalter, M.; Zimmermann, U.; Benz, R. Biophys. J. 1993, 64, 121-128.

(19) Cheng, J.; Sheldon, E. L.; Wu, L.; Uribe, A.; Cerrue, L. O,, Carrino, J.; Heller, M. J.; O'Connell, J. P. Nat. Biotech, 1998, 16, 541-546.

(20) Cheng, Y.; Sheldon, E. L.; Wu, L.; Heller, M. J.; O'Connell, J. P. Anal. Chem. 1998, 70, 2321-2326.

(21) Lee, S.; Tai, Y. Sensors and Actuators A. 1999, 73, 74-79.

(22) McClain, M. A.; Culbertson, C. T.; Jacobson, S. C.; Ramsey, J. M. In Micro Total Analysis Systems 2001; Ramsey, J. M.; van den Berg, A. Eds.; Kluwer Academic Publishers: London, 2001 pp. 301-302.

(23) Nolkrantz, K.; Farre, C.; Brederlau, A.; Kalsson, R. I. D.; Bremian, C.; Eriksson, P. S.; Weber, S. G.; Sandberg, M.; Orwar, O. Anal. Chem. 2001, 73,4469-4477.

(24) Ryttsen, F.; Farce, C.; Brennan, C.; Weber, S. G.; Nollcrantz, K.; Jardemark, K.; Chiu, D. T.; Orwar, O. Biophys. J. 2000, 79, 1993-2001.

(25) Krueger, M.; Thom F Biophys J. 1997,73,2653-2-666.

(26) Owen, D. G.; Piotrowski, M -R. C. J. Physiol 1987, 390,14.

(27) Atkins, .R.; Wang, D.; Burke, R. D. Biotcech. 1999, 27, 94-100.

(28) Huang, Y.; Rubinsky, B. Biomed. Microdev. 1999, 2,145-150.

(29). Huang, Y.; Rubinsky, B. Sens. Actuators A 2001, 89, 242-249.

(30) Morris, C. E. In Cell Physiology Source Book; Sperelakis, N., F.d.; Academic Press: San Diego, 1995; pp 483-489.

(31) Behbehani, M. M. In Cell Physiology Source Book; Sperelakis, N., Ed.; Academic Press: San Diego, 1995; pp 490-494.

(32) Burridge, K.; Chrzanowska-Wodnicka, M. Annu. Rev. Cell Dev. Biol. 1996,12, 463-519.

(33) Chiquet, M.; Matthisson, M.; Koch, M.; Tannheimer, M; Chiquet-Ehrismann, R. Biochem. Cell Biol. 1996, 74, 737-744.

(34) Ingber, D. E. Annu. Rev. Physiol. 1997, 59, 575-99.

(35) Teruel, M. N.; Meyer, T. Biophys. J. 1997, 73, 1785-1796.

(36) Rossignol, .D. P.; Decker, U. L.; Lenzxxarz, W. J.; Tsong, T. Y.; Tcissie, J. Biochem. Biophys. Acta 1983, 763, 346-355.

(37) Tekle, E.; Astamian, R. D.; Chock, P. B. Biochem. Biophys. Res. Commun. 1990, 172, 283-287.

(38) Tekle, E.; Astumian, R. D.; Chock, P. B. Proc. Nad. Acad. Sci. USA 1991, 88, 423-4234. (39) Charlot, G.; Barloz-Lambling, J.; Tremillion, B. Electrochemical Reactions Elsevier Publ. Co.: New York. 1962.

(39) Charlot, G.; Barloz-Lambling, J.; Tremillion, B. Electrochemical Reactions Elsevier Publ. Co.: New York. 1962.

(40) Xue and Yeung, 1996, J. Chromat B 677:233;

(41) Chen., Y., Pepin, A. 2001. Nanofabrication: Conventional and nonconventional methods. Electrophoresis 22:187-207.

(42) Kapur, R., Calvert, J. M., Rudolph, A. S. 1999. J. Biomechanical Eng. 121:65-72.

(43) Kane, R. S., Takayama, S., Ostuni, E., Ingber, D. E., Whitesides, G. M., 1999, Patterning proteins and cells using soft lithography. Biomaterials. 20:2363-76.

(44) Hu et. al 2002, Analytical Chemistry, 74: 4117-4123.

(45) Folch, A., Toner, M. 2000 Annual Reviews of Biomedical Engineering. 2:227-256; 1999 Biomaterials. 20:2363-2376.

(46) Craighead, H. G., James, C. D., Turner, A. M. P. 2001 Current Opinion in Solid State & Materials Science. 5:177-184;

(47) Jung, D. R., Kapur, R., Adams, T., Giuliano, K. A., Mrksich, M., Craighead, H. G., Taylor, D. L. 2001 Critical Reviews in Biotechnology. 21:111-154.

(48) Whitesides, G. M., Ostuni, E., Takayama, S., Jiang, X., Ingber, D. E. 2001 Annual Reviews of Biomedical Engineering. 3:335-375.

(49) Travis E R, Wightman R M 1999, Annu Rev Biophys Biomol Struct 27:77-103.

(50) Suaud-Chagny M F, Cespuglio R, Rivot J P, Buda M, Gonon F 1993, J Neurosci Methods 48:241-250.

(51) Haas, K., Sin, W. C., Javaherian, A., Li, Z., Cline, H. T. 2001. Neuron 29:583-591.

What is claimed is:

1. A cell chamber for electrical lysis of a biological cell comprising,
a transparent substrate material having a bottom and a top surface;
a transparent electrode on the top surface of the transparent substrate material; and
a cell adhesion material on top of the transparent electrode.

2. The cell chamber of claim 1 further including a barrier wall above the top surface and surrounding at least a portion of the cell chamber to form a fluid containing reservoir above the top surface.

3. The cell chamber of claim 1 further including a conductive lead configured to put the transparent electrode in conductive contact with an electrical power supply.

4. The cell chamber of claim 1 wherein the transparent electrode is comprised of indium tin oxide.

5. The cell chamber of claim 1 wherein the transparent electrode is comprised of a layer of metal less than about 200 angstroms in thickness.

6. The cell chamber of claim 1 wherein the transparent electrode is one of an array of separately addressable transparent electrodes configured on the cell chamber.

7. The cell chamber of claim 6 further including a transparent non-conductive layer on top of the substrate material and between the transparent electrodes in the array.

8. The cell chamber of claim 7 wherein the non-conductive layer is made from a material selected from the group consisting of silicon nitride and PDMS.

9. The cell chamber of claim 7, wherein the cell adhesion material is a hydrophilic region above the transparent electrodes in the array.

10. The cell chamber of claim 9 wherein the hydrophilic layer is comprised of polylysine.

11. The cell chamber of claim 7 further including a hydrophobic region above the non-conductive layer and between the electrodes in the array.

12. The cell chamber of claim 11 wherein the hydrophobic region is comprised of a layer of PEG.

13. The cell chamber of claim 7, wherein the cell adhesion material is a hydrophilic region above the electrodes in the array and further including a hydrophobic region above the con-conductive layer and between the electrodes in the array.

14. A cell chamber for electrical lysis of a biological cell comprising,
a substrate material having a bottom and a top surface;
an electrode deposited on the top surface of the substrate; and
an array of cell adhesion materials comprised of a plurality of hydrophilic regions above the electrode, the plurality of hydrophilic regions being separated from one another by adjacent hydrophobic regions.

15. The cell chamber of claim 14 further including a conductive lead configured to put the electrode in conductive contact with an electrical power supply.

16. The cell chamber of claim 15 in a system wherein the power supply is an AC power source.

17. The cell chamber of claim 15 in a system wherein the power supply is a DC power source.

18. The cell chamber of claim 14 wherein the electrode is transparent.

19. The cell chamber of claim 18 wherein the electrode is comprised of indium tin oxide

20. The cell chamber of claim 18 wherein the electrode is comprised of a layer of metal less than about 200 angstroms in thickness.

21. The cell chamber of claim 14 wherein the electrode is one of an array of separately addressable electrodes configured on the cell chamber.

22. The cell chamber of claim 21 wherein the hydrophilic regions are comprised of polylysine.

23. The cell chamber of claim 21 further including a transparent non-conductive layer on top of the substrate material and adjacent to the electrodes in the array.

24. The cell chamber of claim 23 wherein the non-conductive layer is made from a material selected from the group consisting of silicon nitride and PDMS.

25. The cell chamber of claim 14 wherein the hydrophilic regions are comprised of polylysine.

26. The cell chamber of claim 14 wherein the hydrophobic region is comprised of a layer of PEG.

27. A cell chamber for electrical lysis of a biological cell comprising,
a substrate material for receiving a plurality of cells, the substrate material having a top surface and a bottom surface; and
an array of openings through the substrate material between the top surface and the bottom surface of the substrate material.

28. The cell chamber of claim 27 wherein the array of openings are sized to be less than a length of the biological cell.

29. The cell chamber of claim 27 further comprising an electrode located beneath the bottom surface of the substrate.

30. The cell chamber of claim 29 further including a space between the electrode and the bottom surface, the space defining a volume for receiving an electrolyte.

31. The cell chamber of claim 29 wherein the electrode is moveably positionable beneath the array of openings.

32. The cell chamber of claim 27 wherein the array of openings are separated from one another by adjacent hydrophobic regions on the substrate material.

33. A cell chamber for electrical lysis of a biological cell comprising,
a substrate material having a bottom and a top surface;
an array of separately addressable electrode pairs on the top surface of the substrate, each of the electrode pairs comprising a first electrode and second electrode of opposite polarity of the first electrode, the first and second electrodes being separated from one another by a distance of about 200 microns or less;
an array of cell adhesion materials comprised of a plurality of hydrophilic regions separated from one another by adjacent hydrophobic regions and positioned above the array of electrode pairs so that the plurality of hydrophilic regions are located above the plurality of electrode pairs.

34. The device of claim 33 further including a pair of conductive leads for each pair of electrodes in the array for putting the pair of electrodes in electrically conductive contact with a power supply.

35. The cell chamber of claim 33 wherein the electrode pair is transparent.

36. The cell chamber of claim 35 wherein the electrode pair is comprised of indium tin oxide

37. The cell chamber of claim 35 wherein the electrode pair is comprised of a layer of metal less than about 200 angstroms in thickness.

38. The cell chamber of claim 33 wherein the hydrophilic regions are comprised of polylysine.

39. The cell chamber of claim 33 further including a transparent non-conductive layer on top of the substrate material and adjacent to the electrodes in the array.

40. The cell chamber of claim 39 wherein the non-conductive layer is made from a material selected from the group consisting of silicon nitride and PDMS.

41. The cell chamber of claim 33 wherein the hydrophobic region is comprised of a layer of PEG.

42. A device for electrical lysis of a biological cell and collection of at least a portion of contents thereof comprising,
a micro capillary electrophoresis tube having an exterior wall encircling an interior lumen, a distal end for contacting an electrolyte and a proximal end for collecting at least a portion of the contents of a lysed cell, the proximal end having an outer diameter of less than about 200 microns; and
an electrode pair attached to the proximal end of the capillary electrophoresis tube, the electrode pair comprising a positive terminal and a negative terminal separated from one another by distance of about 200 microns or less.

43. The device of claim 42 further including a conductive lead for each electrode in the electrode pair to put the electrodes in electrically conductive contact with an electrical power supply.

44. The device of claim 43 in a system wherein the power supply is an AC power supply.

45. The device of claim 43 in a system wherein the power supply is a DC power supply.

46. The device of claim 43 further comprising an insulating material surrounding the conductive leads.

47. The device of claim 42 wherein the proximal end is tapered.

48. A device for lysis of a biological cell and collection of at least a portion of contents thereof comprising,
a capillary electrophoresis tube having an exterior wall encircling an interior lumen, the interior wall having a thickness of about 200 microns or less,
an electrode pair attached to a proximal end of the capillary electrophoresis tube, the electrode pair comprising a first electrode located on the exterior surface of the exterior wall and a second electrode located on the interior surface of the capillary wall, the first and second electrodes being separated from one another by a distance of about 200 microns or less.

49. The device of claim 48 further including a conductive lead for each electrode in the electrode pair to put the electrodes in electrically conductive contact with an electrical power supply.

50. The device of claim 49 further comprising an insulating material surrounding the conductive leads.

51. The device of claim 48 wherein the proximal end is tapered.

52. A device for lysis of a biological cell and collection of at least a portion of contents thereof comprising,
a capillary electrophoresis tube having an exterior wall encircling an interior lumen, a proximal end at one end of the tube for collecting a sample and a distal end opposite the proximal end;
a conductive wire removably insertable into lumen at the distal end to thereby put the conductive wire in conductive contact with an electrolyte when the electrolyte is within the lumen.

53. A device for facilitating lysis of a biological cell and collection of at least a portion of contents thereof comprising,
a micro capillary electrophoresis tube having an exterior wall encircling an interior lumen, a distal end for contacting an electrolyte and a proximal end for collecting at least a portion of the contents of a lysed cell, the proximal end being tapered and having an outer diameter of about 200 microns or less;
an electrically conductive material deposited on the proximal end of the capillary electrophoresis tube, and
a conductive lead extending down at least a portion of the length of capillary electrophoresis tube and contacting the electrically conductive material.

54. A system for electrical lysis of a biological cell and collection of at least a portion of the contents thereof, comprising
electrical means to lyse the biological cell within 1 second or less of application of an electrical potential to the electrical means; and
microcollection means configured to collect at least a portion of the contents of the lysed cell within a period of about 1 second or less from lysing the cell.

55. A system for electrical lysis of a biological cell and collection of at least a portion of the contents thereof, comprising.
a first electrode that is at least one of positioned on a substrate or positionable within about 200 microns or less from a biological cell in contact with the substrate;
a second electrode that is at least one of positioned on the substrate within about 200 microns of the first electrode or positionable within about 200 microns or less of the first electrode, the first and second electrodes thereby being configured for positioning a biological cell therebetween;
a micro-collection device having a proximal end configured to capture at least a portion of the contents of the biological cell and being at least one of positioned or positionable within about 200 microns of at least one of the first and second electrodes and configured to collect at least the portion of the contents of the biological cell within less than about 1 second of lysis, with the proviso that the micro-collection device is not configured to hold the biological cell within the micro-collection device or to be in contact with the biological cell.

56. The system of claim 55 further including a first conductive lead configured to put the first electrode in conductive contact with an electrical power source to charge the electrode with a first polarity; and
a second conductive contact configured to put the second electrode in conductive contact with the electrical power source to charge the second electrode with a second polarity opposite to the first polarity.

57. The system of claim 55 further comprising a position selection device for positioning the proximal end of the micro collection device in proximity to a selected position on the substrate.

58. The system of claim 55 further comprising a device to initiate collection of the cell contents by the micro collection device within a selected duration of charging the first and second electrodes with an electrical charge.

59. The system of claim 55 wherein the micro collection device is micro capillary electrophoresis tube.

60. The system of claim 55 wherein the first electrode is located on the substrate and the second electrode is located on the micro collection device.

61. The system of claim 55 wherein the first and second electrodes are located on the substrate.

62. The system of claim 55 wherein the first and second electrodes are located on the micro-collection device.

63. The system of claim 55 wherein the micro-collection device is comprised of a plurality of micro-capillary electrophoresis tubes arranged in an array and each micro capillary electrophoresis tube is positioned at a different selectable position above the substrate.

64. The system of claim 55 further including a micro deposit device that is at least one of positioned or positional within 200 microns of at least one of the first electrode, the second electrode and the micro-collection device and wherein the micro deposit device is configured to deposit a test material in proximity to the biological cell when located on the substrate.

65. The system of claim 55 wherein the first electrode is located on a transparent substrate having a bottom and a top surface,
wherein the first electrode is a transparent electrode on the top surface of the transparent substrate ; and
wherein a cell adhesion material is located on top of the transparent electrode.

66. The system of claim 55 wherein the first electrode is located on the substrate that has a bottom and a top surface;
wherein the first electrode is deposited on the top surface of the substrate; and
wherein an array of cell adhesion materials comprised of a plurality of hydrophilic regions are located above the first electrode, the plurality of hydrophilic regions being separated from one another by adjacent hydrophobic regions.

67. The system of claim 55 wherein the substrate has a top surface and
wherein the substrate includes an array of openings through the substrate between the top surface and the bottom surface of substrate.

68. The system of claim 55 wherein both the first and second electrodes are located on the substrate and the substrate has a bottom and a top surface;
wherein the first and second electrodes are arranged in array of separately addressable electrode pairs on the top surface of the substrate, each of the electrode pairs comprising a positive terminal and a negative terminal separated from one another by a distance of about 1 to about 100 microns, and
wherein an of cell adhesion materials comprised of a plurality of hydrophilic regions separated from one another by adjacent hydrophobic regions are positioned above the array of electrode pairs so that the plurality of hydrophilic regions are located above the plurality of electrode pairs.

69. The system of claim 55 wherein the micro-collection device is comprised of a micro capillary electrophoresis tube having an exterior wall encircling an interior lumen, a distal end for contacting an electrolyte and a proximal end for collecting at least a portion of the contents of a lysed cell, the proximal end having an outer diameter of less than about 40 microns; and
wherein the first and second electrodes comprise a pair of electrodes attached to the proximal end of the capillary electrophoresis tube and separated from one another by distance of about 1 to 40 microns.

70. The system of claim 55 wherein the micro-collection device is comprised of a micro capillary electrophoresis tube having an exterior wall encircling an outer lumen located between the exterior wall and an interior wall, the interior wall encircling an interior lumen, the interior wall being separated from the exterior wall by a distance, the capillary electrophoresis tube having a distal end for contacting an electrolyte and a proximal end, the proximal end having an outer diameter of less than about 40 microns and at least one of the first and second lumen on the proximal end is configured to collect the contents of a lysed cell; and
wherein the first and second electrodes comprise an electrode pair attached to the proximal end of the capillary electrophoresis tube, and wherein the first electrode is located on at least one of the exterior wall and the interior wall, and second electrode is located on the other of the exterior wall or interior wall that is not attached to the first electrode.

71. The system of claim 55 wherein the first electrode is located on the substrate and the second electrode is located on the micro-collection device, wherein the micro-collection device is a micro capillary electrophoresis tube having an exterior wall encircling an interior lumen, a distal end for contacting an electrolyte and a proximal end for collecting at least a portion of the contents of a lysed cell and the proximal end is tapered and has an outer diameter of less than about 40 microns;
wherein the second electrode is comprised of an electrically conductive material deposited on the proximal end of the capillary electrophoresis tube, and
wherein a conductive lead extends down a portion of the length of capillary electrophoresis tube and contacts the electrically conductive material.

72. A method of electrical lysis of a biological cell and collection of at least a portion of the contents thereof, comprising
depositing a the biological cell on a substrate;
providing electrical means to lyse the biological cell on the substrate within 1 second or less of application of an electrical potential to the electrical means; and
collecting at least a portion of the contents of the lysed cell with a micro-collection means configured to collect the at least a portion of the contents of the lysed cell within a period of about 1 second or less from lysing the cell.

73. The method of claim 72 wherein the cell is an adherent cell.

74. The method of claim 72 wherein the cell is a non-adherent cell.

75. A method of electrical lysis of a biological cell and collection of at least a portion of the contents thereof, comprising.
depositing the biological cell on a substrate,
providing a first electrode that is at least one of positioned on the substrate less than 200 microns or positionable less than 200 microns from a biological cell in contact with the substrate;
providing a second electrode that is at least one of positioned on the substrate within less than 200 microns from the first electrode or positionable within less than 200 microns from the first electrode, the first and second electrodes;
providing a micro-collection device having a proximal end configured to capture at least a portion of the contents of the biological cell and being at least one of positioned or positionable within less than 200 microns of the first and second electrodes; and
collecting at least the portion of the contents of the biological cell within less than about 1 second of lysis, with the proviso that the cell is not located within the micro-collection device

76. The method of claim 75 wherein the cell is an adherent cell.

77. The method of claim 75 wherein the cell is a non-adherent cell.

78. A method of electrical lysis of a biological cell and collection of at least a portion of the contents thereof, comprising.
depositing the biological cell on a substrate,
providing at least one of a cell chamber according to any one of claims 100-400 and a device according to claims 42-53 to lyse the biological cells using an electrical potential;
lysing the biological cell; and
collecting the at least a portion of the contents of the biological cell with a micro collection device.

79. The method of claim 78 wherein the cell is an adherent cell.

80. The method of claim 78 wherein the cell is a non-adherent cell.
